(19) United States
(12) Patent Application Publication
(10) Pub. No.: US 2011/0105342 A1
(43) Pub. Date: May 5, 2011

(54) METHOD FOR DETERMINING REDUCED PREDISPOSITION TO CANCER BASED ON GENETIC PROFILE

(76) Inventors: Jan Lubinski, Szczecin (PL); Tadeusz Debniak, Mierzyn (PL); Grzegorz Kurzawski, Szczecin (PL); Anna Jakubowska, Szczecin (PL); Krzystof Medrek, Goleniow (PL); Cezary Cybulski, Wolczkowo (PL); Jacek Gronwald, Koszalin (PL); Tomasz Huzarski, Szczecin (PL); Bohdan Gorski, Szczecin (PL); Tomasz Byrski, Szczecin (PL); Joanna Matyjasik, Denatrion (PL); Pablo Serrano Fernandez, Szczecin (PL)

(21) Appl. No.: 12/922,405

(22) PCT Filed: Mar. 13, 2009
(86) PCT No.: PCT/PL2009/000023

§ 371 Date: Dec. 10, 2010

Related U.S. Application Data
(60) Provisional application No. 61/069,403, filed on Mar. 13, 2008.

(51) Int. Cl. C40B 30/00; C12Q 1/68; C40B 40/08

(52) U.S. Cl. 506/7; 435/6; 506/17

(57) ABSTRACT

The invention provide methods for early detection of a reduced risk of developing cancer, which comprises detecting the absence of a series of genetic polymorphisms associated with a predisposition of developing cancer, including the polymorphisms of the genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267, in a biological sample from the analyzed subject, wherein the absence of the genetic polymorphisms is indicative of significantly decreased risk of developing, at least, breast cancer.

### FIELD OF THE INVENTION

Mode and composition for determining the presence of a genetic profile a human being, which is characteristic for a greatly reduced risk of developing cancer. Generally, the invention concerns a new method to estimate life-time risk of developing a tumour, depending on a particular constitutional genotype, composed of a series of different genetic variants of several genes. The subject of the invention allows the identification of particular combinations of genetic variants associated with a protective effect for a particular cancer type and also within particular subgroups of subjects.

### BACKGROUND OF THE INVENTION

Constitutional mutations are a major factor responsible for increased predisposition to different types of cancer. Some of them are high risk factors, such as most mutations in the BRCA1 gene (Ford et al. Am J Hum Genet 1998; 62:676-89; Narod et al. Am J Hum Genet 1995; 56; 254-64; Narod et al. Am J Hum Genet 1995; 57:957-8), others are moderate to low risk factors that increase the risk just slightly, but statistically significant. Such moderate to low risk factors include for example BRCA2, CARD15 (NOD2), CHEK2, CDKN2A (P16), CYP1B1, FGFR2 (KGFR2), MAP3K1 (MEKK1), p53 (TP53), Rs6983267, TNRC9 and XPD (ERCC2), as most outstanding among several others. Without loss of generality, we have focused on constitutional changes of these particular eleven moderate to low risk genes and BRCA1.

Carriers of different mutations of the gene BRCA2 have an increased risk of developing cancer at several sites (Risch et al. J Natl Cancer Inst 2006; 98:1694-706; Antoniou et al. Am J Hum Genet 2003; 72:1117-30). The increase in cancer risk is highly variable and may range from high risk (100-fold for male breast cancer) to moderate risk (7-fold for ovarian cancer and pancreatic cancer, 5-fold for female breast cancer). The effect is also largely dependent on the particular mutation and so, for instance polymorphism C5972T is a rather low risk marker increasing just 1.4-fold the risk of developing breast cancer and just for early onset cases (Gorski et al. Breast Cancer Res 2005; 7:R1023-7).

The 3020insC allele of the gene CARD15/NOD2 has been shown to be significantly associated with increased risk of cancer of different sites (Lubiński et al. Her Can in Clin Pract 2005; 3:59-63; Huzarski et al. Breast Cancer Res Treat. 2005; 89:91-3). CARD15/NOD2 induces a low increase in cancer risk, maximally 2-fold for early-onset breast cancer.

The gene CDKN2A is significantly associated with risk increase for cancer of different sites, either for some of its constitutional changes like A148T allele (Debniak et al. Breast Cancer Res Treat. 2007; 103:355-9; Debniak et al. 2006; 118:3180-2) or its degree of protein expression dependent on promoter methylation (Hsu et al. 2007; 213:412-9; Nakayama et al. 2007; 27:3367-70). The increase in cancer risk is generally low and ranges from 2-fold for lung cancer and 1.4-fold for breast cancer.

Several mutations of the gene CHEK2 have been shown to be significantly associated with increased risk of cancer of different sites (Cybulski et al. Am J Hum Genet 2004; 75:1131-5). The increase in cancer risk ranges from moderate (5-fold for the association of protein truncating alleles with kidney cancer) to low (1.4-fold for the association of protein truncating alleles with breast cancer).

Different single polymorphisms and haplotypes of the gene CYP1B1 have also been suggested to increase the risk of developing a tumour at several sites (Cussenot et al. J Clin Oncol 2007; 25:3596-602; Matyjasik et al. 2007; 106:383-8; Bethke et al. BMC Cancer 2007; 7:123; Justenhoven et al. Breast Cancer Res Treat 2007 Oct. 6, ahead of print). The increase in cancer risk is always rather low; close to 1.5-fold for colorectal cancer, for prostate cancer and for breast cancer.

Germline alterations of FGFR2/KGFR2 have been recently shown to be associated to breast and ovarian cancer (Hunter et al. Nat Genet 2007; 39:870-4; Huijts et al. Breast Cancer Res 2007; 9:R78; Easton et al. Nature 2007; 447:1087-93), with a 1.3-fold increment in the risk. However literature data showing an association between somatic changes of FGFR2 or changes in the levels of its genetic expression, suggest that germline FGFR2 mutations are most probably involved in the aetiology of many other tumours (Yoshino et al. Int J Oncol 2007; 31:721-8; Chaffer et al. Differentiation 2007; 75:831-42; Kwabi-Addo et al. Endocr Relat Cancer 2004; 11:709-24; Lazcoz et al. 2007; 174:1-8; Cho et al. Am J Pathol 2007; 170:1964-74).

Analogously, alterations of the gene MAP3K1/MEKK1 have been demonstrated to be associated with breast and ovarian cancer (Huijts et al. Breast Cancer Res 2007; 9:R78; Easton et al. Nature 2007; 447:1087-93). The association is, though significant, very small (1.1-fold risk increase). Literature data suggest that MAP3K1 is involved in the aetiology of other tumours (Oida et al. Mol Cancer Ther 2007; 6:1440-9; Kim et al. Neurosci Lett 2007; 413:132-6; Zhang et al. J Biol Chem 2004; 279:22118-23; Warmka et al. J Biol Chem 2004; 279:33085-92).

The association between p53/TP53 and cancer has been widely studied for most tumour sites in most human ethnic groups (Varley, Hum Mutat 2003; 21:313-20; Royds et al. Cell Death Differ 2006; 13:1017-26, Savage et al. Pediatr Blood Cancer 2007; 49:28-33, Ueda et al. Gynecol Oncol 2006; 100:173-8; Ignaszak-Szczepaniak et al. Oncol Rep 2006; 16:65-7; Wang-Gohrke et al. Br J Cancer 1999; 81:179-83; Wu et al. Cancer Res 2006; 66:8287-92). Different single polymorphisms and haplotypes are associated with different risk increments. The risk for Li-Fraumeni syndrome (multisite cancer syndrome) increases 100-fold for men and 1000-fold for women. For osteosarcoma the risk may increase up to a moderate 7-fold, but is rather lower for other cancer sites: 3-fold for adrenocortical cancer or 2-fold for sporadic endometrial and ovarian cancer.

Polymorphisms of the genetic marker Rs6983267 have only recently been put in the context of development of cancer of different sites (Cheng et al. Eur J Hum Genet. 2008 Epub ahead of print; Tuupanen et al. Cancer Res 2008; 68:14-7; Halman et al. Nat Genet 2007; 39:954-6; Robbins et al. Genome Res 2007; 17:1717-22; Yeager et al. Nat Genet 2007; 39:645-9; Wokolorczyk et al. Cancer Res, submitted). The risk increment for developing cancer ranges is always below odds ratio 2, being for instance 1.2-fold for colorectal cancer and for breast cancer.

Polymorphisms of the gene TNRC9 have been demonstrated to be associated with breast and ovarian cancer (Huijts et al. Breast Cancer Res 2007; 9:R78; Easton et al. Nature 2007; 447:1087-93, Stacey et al. Nature Genetics 2007; 39:865-9). The association, though significant, implies just a low risk increase ranging from 1.2-fold to 1.6-fold depending on the particular polymorphism and studied group.

Similarly, common polymorphisms of the gene XPD/ERCC2 are known to be associated tumours at different sites (Debniak et al. Breast Cancer Res Treat 98:209-15; Lopez-Cima et al. BMC Cancer 2007; 7:162; Chen et al. Carcinogenesis 2007; 28:2160-5; Andrew et al. Hum Hered 2008; 65:105-18; Bau et al. Anticancer Res 2007; 27:2893-6; Shao et al. Cancer Genet Cytogenet 2007; 177:30-6). The risk increments are low and range from 1.8-fold for prostate cancer and 1.5-fold for breast cancer.

Summarising, there is a set of at least 11 genetic markers associated with a moderate to low increase in the risk of developing cancer at different sites, and one high risk genetic marker (BRCA1). They have been validated in different populations and all of them share breast cancer as one of the tumours they are associated with.

Most of the 12 genetic markers we have focused in, have been independently subject of several patents where the detection each particular genetic marker serves as an indicator for assessment of cancer risk, for introduction of prophylactic measures and sometimes for prognosis of disease outcome after cancer diagnosis. Specifically for breast cancer we may list WO2005121786, WO03104474, US2004014115, US2005019782, WO9605308, U.S. Pat. No. 6,514,713 and US2005019782 for BRCA1; WO9915701, WO9915704, WO9928506, WO9909164, WO03068054, U.S. Pat. No. 6,033,857, US2004115717, US2006154272 and US2002031785 for BRCA2; US2005191669 WO2005068659 for CARD15; US2005191669 WO2005068659 for CDNK2A; PL367319, US2005191669 and WO2005068659 for CHEK2; WO2006137751 and US2007009943 for CYP1B1.

In the current polygenic model for cancer it is assumed that the co-occurrence of several factors that are otherwise associated to a moderate or low risk when taken separately, may turn to a high risk factor when present as a combination (Tyrer et al. Genet Epidemiol 2006; 30:636-43; Pharoah et al. PLoS Genet 2007; 3:e42). This paradigm is actually mostly burdened by a lack sufficient statistical power to detect these with appropriate levels of statistical significance, due to the large amounts of studied subjects needed to perform such analyses.

Nevertheless, first studies have already managed to demonstrate a high-risk association between a combination of different markers and the risk of breast cancer (Cox et al. BMC Cancer. 2006; 6:217; Onay et al. BMC Cancer 2006; 6:114; Xu et al. Carcinogenesis 2007; 28:1504-9; Spurdle et al. Cancer Epidemiol Biomarkers Prey 2007; 16:769-74; Hong et al. Cancer Epidemiol Biomarkers Prey 2007; 16:1784-94; Briollais et al. BMC Med 2007; 5:22; Hsu et al. Cancer Epidemiol Biomarkers Prey 2007; 16:2024-32), bladder cancer (Andrew et al. Hum Tiered 2008; 65:10518; Chen et al. Carcinogenesis 2007; 28:2160-5; Manuguerra et al. Carcinogenesis 2007; 28:414-22), lung cancer (Vogel et al. Mutat Res 2007 November; Epub ahead of print; Hsu et al. J Pathol 2007; 213:412-9; Tsou et al. Mol Cancer 2007; 6:70; Manuguerra et al. Carcinogenesis 2007; 28:414-22), gastric cancer (Boccia et al. BMC Cancer 2007; 7:206), glioma (Liu et al. Hum Mutat 2007 December; Epub ahead of print) and leukaemia (Manuguerra et al. Carcinogenesis 2007; 28:414-22) among others. Some among them have also succeeded including environmental factors in the multifactorial model.

Analogously, the same approach has been applied with success in the field of gene expression for the detection of cancer (Yue et al. Hepatobiliary Pancreat Dis Int 2002; 1:309-11) and breast cancer (Bolufer et al. Clin Chim Acta 1994; 229:107-22) and in the field of prediction of responsiveness towards anticancer therapy (Apetoh et al. Immuno) Rev 2007; 220:47-59).

The multifactorial model does not presume a particular type of effect derived from the presence of multiple markers of cancer risk. Some effects may be just additive, while others may be synergistic and thus implying some kind of interaction (directly or mediated by other genetic products) between the involved markers. As an example of the latter, and without loss of generality, one may consider the case of CYP1A1 and CYP1B1. A particular polymorphism of CYP1A1 significantly decreases the risk of developing lung cancer, while when present in combination with a particular polymorphism of CYP1B1, the risk increases over 2-fold (Yoon et al. Lung Cancer 2007 November; Epub ahead of print).

The multifactorial model of cancer has also been subject of a patent application focused on three genes, CYP1B1, CHEK2 and BRCA2 (WO2007148997). The determination of the genes as indicators for inherited predisposition to cancer had already been patented for each of the markers separately. Patent WO2005068659 had claimed the identification of the protein truncating variants IVS2+IG>A and 11OOdelC as well as the missense variant I157T of CHEK2 as indicators of increased colorectal and prostate cancer risk. Analogously, screening panels for several BRCA2 variants designed for the evaluation of breast and ovarian cancer risk had been subject of several patents (WO9915701; WO9915704; WO9928506; WO9909164; WO03068054; U.S. Pat. No. 6,033,857; US2004115717; US2006154272; US2002031785). The determination of the same haplotype variants of CYP1B1 for evaluation of cancer risk stood also under patent protection (WO2006137751, US2007009943). Nevertheless, in patent application WO2007148997 authors demonstrated that the effect of the combined presence of all three genetic markers of CYP1B1, CHEK2 and BRCA2 in the explored subject largely exceeded the expected by the linear or additive model. In other words, the state of the art on the three markers was not enough to predict the risk increment in a person carrying all three genetic variants at the same time. The basic inventive step relied on the interactive effect that changes the moderate to low cancer risk association of these three markers into a high-risk marker combination, qualitatively and quantitatively different than the sum of all three effects independently.

Similarly, other patents have dealt with the possible regulatory function associated with particular genetic markers in carriers of high to moderate risk mutations (WO01118254). A particular mutation of the gene RAD51 increases additionally the risk of developing a cancer among carriers of (already risk increasing) mutations of BRCA1 or BRCA2. That risk increase is assumed to rely on a direct interaction of the genetic products of RAD51, BRCA1 and BRCA2 respectively.

The subject of the present invention, does not focus on high-risk combinations of low to moderate risk markers, but rather the opposite. However, the rationale is the same as in the cases mentioned above. In the present invention it is shown how the absence of highly specific combinations of genetic markers for cancer risk can be used to determine a protective genetic profile with an outstandingly low predisposition for developing cancer.

To make the difference clear, the combination of all three BRCA2, CYP1B1 and CHEK2 risk markers claimed in WO2007148997 affects roughly 0.6% of all patients and a much lower percentage of the general population. But the absence of that combination does not protect against cancer in the rest of the population, since obviously only among patients we still have 99.4% of individuals affected but not carrying the marker combination. The subject of the present invention, instead, is a conditional selection where patients carrying none of the mutations of the given list are compared to controls carrying none of the mutations. The result is an association between the absence of a series of genetic markers with a highly increased protection against development of cancer. The subject of this invention is best determining such a protective effect when the coverage of the markers in a sample which is large enough to warrant statistical power approaches to 100% in the patient group and the difference is maximized in comparison to the controls group. Low-risk common variants are particularly important for this strategy.

The final list of genetic markers that have to be absent to reduce the risk of developing cancer is highly specific for the chosen patient group or subgroup and is generated in a stepwise process. The genetic marker showing the highest odds ratio between cases and controls is selected first. Carriers for that mutation are then removed from both the cases and the controls group. For the remaining individuals, the process is repeated until the addition of a new marker does not generate a relevant improvement of the odds ratio and/or does not generate a relevant increase in the coverage of the sample of patients.

Without loss of generality, breast cancer patients carrying none of the mutations among BRCA1 (C61G, 4153delA, 5382insC), BRCA2 (T1915M), CHEK2 (1VS+1G/A, I157T, 1100delC, del5395). CDKN2A (A148T), XPD (D312N, K751Q), P53 (R72P), TNRC9 (Rs3803662 non-TT), FGFR2 (Rs1219648 GG) are just 9.4% of the total and healthy controls 16.6%. The odds ratio is 1.9 and is statistically significant.

Without loss of generality and analogously for lobular breast cancer, patients carrying none of the mutations among BRCA1 (C61G, 4153delA, 5382insC), BRCA2 (T1915M), CHEK2 (IVS+1G/A, I157T, 1100delC, del5395), P53 (R72P). TNRC9 (Rs3803662 non-TT), FGFR2 (Rs1219648 non-AA), CARD15 (3020insC), MAP3K1 (Rs889312 non-AA) are just 0.7% of the total and healthy controls 12.1%. The odds ratio is 19.2 and is statistically significant.

Although it expectable that people not carrying any of the risk markers in a given list, are at reduced risk of developing cancer, no one skilled in the art could predict the extension of the coverage in the patients group (close to 100%) nor could he predict the order of magnitude of the protective effect ranging from 2- to 19-fold, which is clearly besides any mere linear effect.

This invention is relevant for different aspects. The determination of a genetic profile indicative for greatly reduced risk of developing cancer finds its application for subjects under carcinogen exposure (e.g. occupational exposure) concerned about the risk of developing cancer given their genetic background. In extreme it may be particularly relevant for some cases of anxiety syndromes derived from such exposure or of psychogenic origin. Moreover, the invention finds application in the frame of Public Health. From a socio-economic point of view it is relevant to know which persons are at greatest risk, as well as which persons have a protective genetic background to optimize the use of resources in large scale monitoring or prevention programs.

In summary, we can conclude that the current state of the art shows an association between germline mutations of different genes—detected by conventional methods—and the risk of developing cancer. It is also state of the art how some combinations of the former genetic markers are also associated with cancer risk in a way which could not be predicted from the association of each of that markers separately. Subject of this invention is a method for predicting a particularly reduced risk of developing cancer, dependent on particular constitutional genotype combinations of a list of markers associated with cancer.

The invention is described in the following examples of the application, to better illustrate its relevance. However, the invention cannot be reduced to the mentioned examples.

### EXAMPLES

It is well recognized that a small percentage of common malignancies that include those of the breast, ovary or colon cancer are a result of constitutional mutations in genes such as BRCA1, BRCA2, MSH2, MLH1 and APC. Genetic predispositions for the majority of tumours, however, remains unknown but evidence is accumulating to suggest that low to moderate penetrance genes contribute to disease risk.

There are several different approaches that have been used to identify low to moderate genetic risk factors and currently the most popular is to perform genome-wide association studies on an appropriately large series of unselected cancer cases and unaffected matched controls (1). These studies have focused on large multi-centre and multi-ethnic cohorts that have lead to the identification of genetic polymorphisms of moderate to low risk that appear to be common in the populations examined (2).

Since the aims of current research into the causes of malignancy is currently engaged in verifying the hypothesis that genetic constitutional changes contribute to the cancer predisposition in general, the use of genome-wide association studies for the identification of low to moderate genetic risk factors does, however, have some important limitations:
a) Pathological and/or clinical characteristics of cancers can be different depending on the associated moderate/low genetic risk markers (3-6). Therefore, it is critical to use an appropriate cancer population that is stratified by age at diagnosis and tumour pathology.
b) As low to moderate risk markers occur with varying frequency in different ethnic groups the power to detect any association is compromised especially for low risk disease modifiers. Ideally, it is preferable to study large homogeneous populations to reduce population variance and increase the likelihood of identifying true genetic associations even when they are considered to be of low penetrance.

Taking the above into account we established an alternative strategy to efficiently identify panels of low-to-moderate genetic breast cancer risk markers using the following criteria:
a. Consecutive breast cancer cases stratified into groups—by age at diagnosis and tumour pathology from homogeneous Polish population,
b. Unaffected controls matched by age, sex, ethnicity and geographic location with negative cancer family histories
c. Using single candidate markers (mutations/polymorphisms) known to be associated with increased genetic predisposition to breast cancers.

Employing the above criteria allowed us to identify a series of markers characteristic for 19 of 20 breast cancer groups that occur in more than 90% and almost 100% of patients in 18 out of the 20 breast cancer groups analyzed. The results provide evidence that there are a series of genetic polymorphisms that predispose to breast cancer and suggest that other markers are likely to exist that are associated with malignancies in general.

Studies were performed on a series of 977 newly diagnosed consecutively collected breast cancer patients (participation rates above 95%) who underwent mastectomy at the Regional Oncology Hospital (Szczecin) in the years 2002, 2003, 2006 and 2007 or the University Hospital SPSK2 (years: 2002-2007), Szczecin, West-Pomerania, Poland.

Between the years 2000 and 2001 family doctors from the region in and around Szczecin collected cancer family history questionnaires from approximately 1.3 million inhabitants.

Persons with a negative cancer family history were invited to participate in the project. A total of 1040 adult females accepted the invitation to participate and from these women the control population was created, which comprised 977 females matched for year of birth (±2 years), ethnicity and location. The 977 pairs were classified into 20 different groups. Finally, the following groups were included for analysis:

1. All consecutive n=977
2. Diagnosed under the age of 51 yrs n=310
3. Diagnosed above the age of 50 yrs n=667
4. Ductal, low grade (I and II degree by Bloom) n=401
5. Ductal, high grade (III degree by Bloom) n=167
6. Lobular n=140
7. ER+ (estrogen receptor positive) n=508
8. ER− (estrogen receptor negative) m=201
9. Ductal, low grade and diagnosed above the age of 50 yrs n=266
10. Ductal, high grade and diagnosed above the age of 50 yrs n=104
11. ER+ and diagnosed above the age of 50 yrs n=334
12. ER− and diagnosed above the age of 50 yrs n=118
13. Ductal, low grade and diagnosed under the age of 51 yrs n=135
14. Ductal, high grade and diagnosed under the age of 51 yrs n=63
15. ER+ and diagnosed under the age of 51 yrs n=174
16. ER− and diagnosed under the age of 51 yrs n=83
17. Ductal, low grade and ER+ n=259
18. Ductal, low grade and ER− n=55
19. Ductal, high grade and ER+ n=76
20. Ductal, high grade and ER− n=73

All participants provided a blood sample from which DNA was extracted for analysis. Molecular studies included the analysis of the mutations/polymorphisms as described in table 1. Experimental conditions to perform ASA, RFLP-PCRs, real time PCR and DNA sequencing have been reported previously (Table 1).

The particular experimental conditions for this example were as follows:

### DNA Isolation

5 ml peripheral blood was obtained from patients and mixed with 100 μl 1M EDTA, then was centrifuged in 50 ml polypropylene tubes by 10 minutes at 3000 g in 4° C. Serum in upper faze was removed, and pellet containing cells was mixed with 45 ml buffer 2× (0.1M NH4Cl, 0.25M KHCO₁, 1 mM EDTA) and was left for 15 minutes in 4° C. Then mixture was centrifuged at 3000 g for 10 minutes in 4° C. Supernatant was removed after centrifugation. The remaining pellet with leukocytes was suspended in 2× buffer and centrifuged 10 minutes at 3000 g in 4° C. This purification of leukocytes in 2× buffer and centrifugation was repeated three times until pure leukocyte pellet was obtained. Then to leukocytes were mixed with 3 ml digestion buffer (50 mM NaCl, 25 mM MgCl₂, 1 mM EDTA; pH 8.0) with 200 μl 10% SDS and 500 μg Proteinase K. Digestion was carried out 24 h in 37° C.

DNA was purified using phenol/chloroform. In brief digestion products was mixed with 3 ml phenol buffered 0.5M Tris HCl (pH 8.4), and then 3 ml chloroform and isoamyl alcohol mixture (mixed in proportion 1:25 vol/vol). Mixture was agitated for about 1 minute and centrifuged 10 minutes at 8000 g in 20° C. After centrifugation upper faze was replaced to new tube, and mixed with equal volume of chloroform and thereafter centrifuged 10 minutes at 8000 g. Above described purification with chloroform was repeated 3-times until protein ring in inter-phase had disappeared.

The purified water faze containing DNA was mixed with 5M NaCl in proportion 10:1 (vol/vol) and 96% ethanol in the proportion of water phase with NaCl to ethanol 1:10 (vol/vol). Mixture was left overnight in 20° C. The resultant DNA pellet was placed in a new tube and purified with 70% ethanol, centrifuged at 3000 g for 5 minutes, and ethanol was poured out. Then purified DNA pellet was dried in open tube for 30 minutes at 37° C. DNA resuspended in 400 μl TE buffer (25 mM Tris HCl, 1 mM EDTA: pH 8.4) was stored in 4° C. until use.

### 1. BRCA1

### Multiplex ASO-Polymerase Chain Reaction

Variants C61G, 4153delA and 5382insC

The reaction mixture includes a mixture of primers responsible for
amplification of a fragment of exon 5 enclosing the location of the eventual mutation C61G. Additional PCR products are indicators for the quality of the PCR reaction and serve as internal controls. Restriction enzyme AvaII cuts the PCR product of exon 5 into two smaller fragments, whenever mutation C61G is present,
amplification of a fragment of exon 11 only in case mutation 4153delA is present in the analyzed material.

amplification of a fragment of exon 20 only in case mutation 5382insC is present in the analyzed material, where the lengths of the PCR products for exons 5, 11 and 20 are chosen to allow for simple and unequivocal identification using electrophoresis in agarose gel.

Primer sets used for the reaction mixture
I B1EX5IK1F, B1EX5IK1R, B1_{—}4154DELAI2F, B1_{—}4154DELAI1R, B1-5382INSCHI1F, B1-53821NSCI1R
II B1EX5IK2F, B1EX5IK2R, B1_{—}4154DELAK2F, B1_{—}4154DELAI2R, B1-5382INSCK2F, B1-53821NSCI2R
III B1EX5IK1F, B1EX5IK2R, B1_{—}4154DELAI2F, B1_{—}4154DELAI2R, B1-5382INSCI2F, B1-5382INSCI1R

| | | | Primer | Primer for |
|---|---|---|---|---|
| | | | for sense | antisense |
| | | | strand | strand |
| Primer pairs | Primer ID | Function | [F] 5′ -> 3′ | [R] 5′ -> 3′ |
| Pair 1 for | B1-5382INSCI1 | identification | CAC TTC CAT | TAC CTT TCT |
| BRCA1 ex. 20 | | | TGA AGG AAG | GTC CTG GGG |
| 5382 ins C | | | CTT C | AT |
| Pair 2 for | B1-5382INSCI2 | identification | TGA CGT GTC | ACC TTT CTG |
| BRCA1 ex. 20 | | | TGC TCC ACT | TCC TGG GGA |
| 5382 ins C | | | TC | TT |
| Pair 3 for | B1-5382INSCK1 | control | CAC TTC CAT | CAA AGG GGA |
| BRCA1 ex. 20 | | | TGA AGG AAG | GTG GAA TAC |
| 5382 ins C | | | CTT C | AG |
| Pair 4 for | B1-5382INSCK2 | control | ATA TGA CGT | CAA AGG GGA |
| BRCA1 ex. 20 | | | GTC TGC TCC | GTG GAA TAC |
| 5382 ins C | | | AC | AG |
| Pair 1 for | B1EX5IK1 | identification/ | CTC TTA AGG | TTC CTA CTG |
| BRCA1 ex. 5 | | control | GCA GTT GTG | TGG TTG CTT |
| 300T→G | | | AG | CC |
| Pair 2 for | B1EX5IK2 | identification/ | ATG GCT CTT | TGT GGT TGC |
| BRCA1 ex. 5 | | control | AAG GGC AGT | TTC CAA CCT |
| 300T→G | | | TG | AG |
| Pair 1 for | B1_4154DELAI1 | identification | CAA AGG CAT | CAA GCC CGT |
| BRCA1 ex. 11 | | | CTC AGG AAC | TCC TCT TTC |
| 4153 delA | | | ATC | TCA |
| Pair 2 for | B1_4154DELAI2 | identification | TTG GCT CAG | AAG CCC GTT |
| BRCA1 ex. 11 | | | GGT TAC CGA | CCT CTT TGT |
| 4153 delA | | | AG | CA |
| Pair 3 for | B1_4154DELAK1 | control | TTG GCT CAG | GTG CTC CCC |
| BRCA1 ex. 11 | | | GGT TAC CGA | AAA AGC ATA |
| 4154 delA | | | AG | AAC |
| Pair 4 for | B1_4154DELAK2 | control | TCC TAG CCC | GTG CTC CCC |
| BRCA1 ex. 11 | | | TTT CAC CCA | AAA AGC ATA |
| 4153 delA | | | TAC A | AAC |

The reaction ASO-PCR was carried out in an automatic thermocycler (DNA ThermalCycler 9600—Perkin Elmer). The mixture of substances for 25 μl volume comprised: 1 μl (50 ng-200 ng) genomic DNA, 2.5 μl reaction buffer (100 mM Tris-HCl, 5500 mM KCL, 15 mM MgCl₂, 1 mg/ml gelatin; pH 8.6), 2-14 μM of each primer, 200 μM of each desoxynucleotide (dATP, dCTP, dGTP and dTTP) and 1 U Taq DNA polimerase. For each reaction there are additionally 3 positive controls (control DNA from carriers of the mutations 5382insC, C16G and 4153delA) and 2 negative controls (control DNA from non-carriers and a control with no DNA at all).

Amplification takes place under the following conditions:
DNA denaturation at 95° C. during 5 minutes,
10 cycles consisting each of
denaturation at 94° C. during 30 seconds
primer binding at 68-58° C. during 30 seconds*
elongation of complementary DNA at 72° C. during 35 seconds
30 cycles consisting each of
denaturation at 94° C. during 30 seconds
primer binding at 57° C. during 30 seconds
elongation of complementary DNA at 72° C. during 30 seconds
   * for the first 10 cycles the temperature for primer binding is decreased in 1.2° C. for each following cycle (in the first cycle it took 68° C., in the second 66.8° C., in the third 65.6° C., in the fourth 64.4° C., in the fifth 63.2° C., in the sixth 62° C., in the seventh 60.8° C., in the eigth 59.6° C., in the ninth 58.4° C. and in the tenth 57.2° C.).

5 μl of PCR reaction products were mixed with 10 μl Stop buffer (Solution of saccharose stained with bromophenol blue) and subjected to electrophoresis in agarose gel (1.5% agarose SeaKem FMC, 1× bufor TBE, 25 μg/ml ethidium bromide) under 6V/cm for 30 min. The separated products in the gel were visualized with UV illumination.

### 2. BRCA2

### Restriction Fragment Length Polymorphism Polymerase Chain Reaction (RFLP-PCR)

Variant C5972T (T1915M)

The C5972T variant (Thr1915Met) was analyzed by restriction fragment length polymorphism PCR using b5972F (5′-CTC TCT AGA TAA TGA TGA ATG ATG CA) and b5972R (5′-CCA AAC TAA CAT CAC AAG GTG) primers. The forward primer introduces an artificial restriction site for the Mph1103I enzyme (Fermentas). PCR products were digested in mutation positive cases. PCR reactions were carried out in DNA ThermalCycler 9600 (Perkin Elmer) in a volume of 25 μl included: 1 μl (50 ng) genomic DNA, 4 pmol b5972F primer, 4 pmol b5972R primer 2.5 μl PCR buffer (100 mM Tris-HCl, 500 mM KCL, 15 mM MgCl2, 1 mg/ml gelatin; pH 8.6), 200 μM each dATP, dCTP, dGTP i dTTP and 1 U Taq DNA polymerase. In each reaction negative control (control without DNA) was used.

### PCR Conditions:

Initial denaturation −95° C. 5 minutes
35 cycles, each of: denaturation −94° C. 30 s
primer annealing −53-58° C. 45 s
   primer elongation −72° C. 1 minute
      elongation −72° C. 1 minute

Digestion was performed overnight at 37° C. in volume of 20 μl containing: 5 μl PCR product, 1*NE Buffer 3 (New England Biolabs) and 2 U Mph1103I enzyme. Then, 151 μl of digestion product was mixed with 10 μl loading buffer and went electrophoresis in agarose gel (2% agarose gel (SeaKem FMC), 1*buffer TBE, 25 μg/ml ethidium bromide) at 6V/cm for 30 minutes. Separated PCR products were visualized in UV light. PCR product was digested in cases with the mutation.

### 3. CARD15

### Restriction Fragment Length Polymorphism Polymerase Chain Reaction (RFLP-PCR)

Variant 3020insC

The 3020insC alteration was identified by RFLP-PCR on 1 μl genomic DNA (˜200 ng) with forward primer (30 pmol/μl) F 5′ TCCGTCTTAGCTGAGTGGCGTAGGCAGAAGCCCTCCTGCAGGGCC 3′, and 0.125 μl reverse primer (30 pmol/μl) R 5′ TCACTGAATGTCAGAATCAGAAG 3′. PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2(Expand Long Template PCR System Roche—22.5 mM MgCl₂), 200 μM each dATP, dCTP, dGTP i dTTP and 1 U Taq DNA polymerase. In each reaction negative control (control without DNA) was used.

### PCR Conditions:

Initial denaturation −94° C. 3 minutes
55 cycles, each of: denaturation −94° C. 30 s
primer annealing −59° C. 30 s
primer elongation −72° C. 30 s
   30 cycles, each of: denaturation −94° C. 30 s
primer annealing −58° C. 30 s
primer elongation −72° C. 30 s
elongation −72° C. 7 minutes

The digestion of the PCR product is based on a restriction enzyme mix composed of 6 μl Water, 1.6 μl 10× buffer B and 0.2 μl ApaI (10 U/μl) Fermentas (ER1411). 7.5 μl of the restriction enzyme mix are added to the PCR product and incubated overnight at 37° C. Then 5 μl loading buffer is added to the digested product and 18-19 μl of the resulting mixture is separated in agarose gel (3%) at 9V/cm for 30 min. The digested product sizes are 200 bp for wild type homozygous, 155 bp for mutated homozygous and 200 bp+155 bp for heterozygous. Separated products were visualized in UV light and genotype assessed for each sample.

### 4. CDKN2A

### Restriction Fragment Length Polymorphism Polymerase Chain Reaction (RFLP-PCR)

### Variant 442G>A (A148T)

The A148T mutation was identified by RFLP-PCR using Sac II restriction enzyme (Eurx). PCR was performed with primers npl6ex2f (AGGGGTAATTAGACACCTGG; SEQ ID NO: 39) and npl6ex2r (TTTGGAAGCTCTCAGGGTAC; SEQ ID NO: 40). PCR reactions was carried out in DNA ThermalCycler 9600 (Perkin Elmer). A volume of 25 ul of reaction mixture included: 1 μl (50 ng) genomic DNA genomic DNA, 4 pmol npl6ex2f primer, 6 pmol npl6ex2r primer, 2.5 μl PCR buffer (100 mM Tris-HCl, 500 mM KCL, 15 mM MgCl2, 1 mg/ml gelatin; pH 8.6), 200 μM each dATP, dCTP, dGTP i dTTP and 1 U Taq DNA polymerase. In each reaction negative control (control without DNA) was used.

PCR conditions:
Initial denaturation −95° C. 5 minutes
10 cycles, each of: denaturation −95° C. 30 s
primer annealing −68-58° C. 40 s
   primer elongation −73° C. 1 minute
      30 cycles, each of: denaturation −95° C. 20 s
primer annealing −57° C. 25 s
   primer elongation −73° C. 1 minute
      elongation −73° C. 1 minute

Digestion was performed overnight at 37° C. in volume of 20 ul containing 5 ul gel PCR product, 1× NE Buffer 4 (New England Biolabs) and 3U Sac II enzyme. Then, 15 ul of digestion product was mixed with 10 ul loading buffer and was electrophoresed in agarose gel (2% agarose gel (SeaKem FMC), 1× buffer TBE, 25 ug/ml ethidium bromide) at 6V/cm for 30 minutes. Separated PCR products were visualized in UV light. PCR product was digested in cases with the wild type. All cases with alterations detected during electrophoresis were sequenced in order to confirm the presence of the A148T change.

### 5. CHEK2

### Restriction Fragment Length Polymorphism Polymerase Chain Reaction (RFLP-PCR)

Variant IVS2+1G>A

The IVS2+1G>A mutation was identified by RFLP-PCR using Hpy 188III (New England Biolabs). PCR was performed with primers CHEK2ex2/3F: 5′-ATTTATGAGCAATTTTTAAAC G-3′ (SEQ ID NO: 35) and CHEK2ex2/3R: 5′-TCCAGTAACCATAAGATAATAATATTA C-3′ (SEQ ID NO: 36). PCR reactions were carried out in DNA ThermalCycler 9600 (Perkin Elmer) in a volume of 25 μl included: 1 μl (50 ng) genomic DNA, 4 pmol CHEK2ex2/3F primer, 4 pmol CHEK2ex2/3R primer 2.5 μl PCR buffer (100 mM Tris-HCl, 500 mM KCL, 15 mM MgCl2, 1 mg/ml gelatin; pH 8.6), 200 μM each dATP, dCTP, dGTP i dTTP and 1 U Taq DNA polymerase. In each reaction negative control (control without DNA) was used.

### PCR Conditions:

Initial denaturation −95° C. 5 minutes
35 cycles, each of: denaturation −94° C. 30 s
primer annealing −53-58° C. 45 s
   primer elongation −72° C. 1 minute
      elongation −72° C. 1 minute

Digestion was performed overnight at 37° C. in volume of 20 μl containing: 5 μl PCR product, 1*NE Buffer 4 (New England Biolabs) and 2 U Hpy188III enzyme. Then, 151 μl of digestion product was mixed with 10 μl loading buffer and went electrophoresis in agarose gel (2% agarose gel (SeaKem FMC), 1*buffer TBE, 25 μg/ml ethidium bromide) at 6V/cm for 30 minutes. Separated PCR products were visualized in UV light. PCR product was digested in cases with the mutation.

### Variant 430T>C (I157T)

The 430T>C variant (Ile157Thr) was analyzed by restriction fragment length polymorphism polymerase chain reaction, using Ch157F (5′-ACCCATGTATCTA GGAGAGCTG-3′ (SEQ ID NO: 37)) and Ch157R (5′-CCACTGTGATCTTCT ATGTCTGCA-3′ (SEQ ID NO: 38)) primers. The reverse primer introduced artificial restriction site for PstI enzyme. The PCR products were digested in mutation positive cases. Experimental conditions were as for RFLP-PCR for the IVS2+1G>A variant of CHEK2 with exception of that 2 U PstI enzyme (instead of Hpy188III) and NE Buffer 3 (instead of NE Buffer 4) were used and RFLP-PCR products were separated in 3% agarose gel.

### Multiplex Polymerase Chain Reaction

Multiplex PCR reactions was carried out in DNA ThermalCycler 9600 (Perkin Elmer) in volume of 25 μl included: 1 μl (50 ng) genomic DNA, 5 pmol CHLdelR primer, 5 pmol CHLcF primer, 5 pmol CHLdel2F primer or, respectively, 5 pmol CHLc2R primer, 5 pmol Chk2ex10f primer, 5 pmol Chk2ex10r primer, 5 pmol Chk2delC primer, 2.5 μl PCR buffer (100 mM Tris-HCl, 500 mM KCL, 15 mM MgCl₂, 1 mg/ml gelatin; pH 8.6), 200 μM each dATP, dCTP, dGTP i dTTP and 1 U Taq DNA polymerase. In each reaction negative control (control without DNA) was used.

5 Variant del5395

Two primers pairs were designed specifically for genotyping of a large deletion of 5395 nucleotides including exons 9 and 10 in multiplex PCR reaction. The first pair (CHLdel2F 5′-TGT AAT GAG CTG AGA TTG TGC-3′; CHLc2R 5′-CAG AAA TGA GAC AGG AAG TT-3′) flanked breakpoint site in intron 8. The second pair (CHLdelR 5′GTC TCA AAC TTG OCT GCG-3′; CHLcF 5′CTC TGT TGT GTA CAA GTG AC-3′) flanked breakpoint site in intron 10.

### PCR Conditions:

Initial denaturation −95° C. 5 minutes
35 cycles, each of: denaturation −94° C. 30 s
primer annealing −53-58° C. 45 s
primer elongation −72° C. 1 minute
   elongation −72° C. 1 minute

In mutation negative cases, only two PCR fragments of 379 and 522 bp were amplified from the wild type allele. The forward primer of the first pair and the reverse primer of the second pair amplified additional PCR product of 450 bp in mutation positive cases.

Variant 1100delC

The 1100delC was analyzed using an allele specific polymerase chain reaction assay using primers Chk2ex10f (5′-TTA ATT TAA GCA AAA TTA AAT GTC) Chk2ex10r (5′-GGC ATG GTG GTG TGC ATC), Chk2delC (5′-TGG AGT GCC CAA AAT CAT A). Multiplex PCR conditions as for variant del5395.

### 6. CYP1B1

### Restriction Fragment Length Polymorphism Polymerase Chain Reaction (RFLP-PCR)

### Variant 355T>G (A119S)

The 355T/T variant alteration was identified by RFLP-PCR using Eam 1105I and PdiI restriction enzyme (Fermentas). PCR was performed with primers e.g. CYP119F (CTCGTTCGCTCGCCTGGCGC) and e.g. CYP119R (GAAGTTGCGCATCATGCTGT). PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2(Expand Long Template PCR System Roche—22.5 mM MgCl), 200 μM each dATP, dCTP, dGTP i dTTP and 1 U Taq DNA polymerase. In each reaction negative control (control without DNA) was used.

### PCR Conditions:

Initial denaturation −95° C. 15 minutes
15 cycles, each of: denaturation −95° C. 30 s
primer annealing −62-54.5° C. 30 s
   (decrease temperature 0.5° C. in each cycle)
   primer elongation −72° C. 2 minutes
   21 cycles, each of: denaturation −95° C. 30 s
primer annealing −57° C. 30 s
primer elongation −72° C. 30 s
   elongation −72° C. 8 minutes

Digestion was performed overnight at 37° C. in volume of 24 μl containing: 12 μl PCR product, 10× Buffer Tango (Fermentas) and 2U Eam 1105I enzyme (Fermentas). Then, 15 μl of digestion product was mixed with 10 μl loading buffer and was electrophoresed in agarose gel (3% agarose gel (SeaKem FMC), 1× bufor TBE, 25 μg/ml ethidium bromide) at 6V/cm for 30 minutes. Separated PCR products were visualized in UV light. PCR product (250 bp) was digested on two fragments: 136 bp and 114 bp in cases which containing nucleotide T in 355 nucleotide site of CYP1B1 gene. All cases with alterations are verified by using PdiI enzyme restriction (Fermentas). Restriction mixture in volume 18 μl containing: 4 μl PCR product, 10× Buffer Tango (Fermentas) and 2U PdiI enzyme (Fermentas). Then, 15 μl digestion product was electrophoresed in the same conditions. PCR product (250 bp) was digested on two fragments: 138 bp and 112 bp in cases which containing nucleotide G in 355 nucleotide site of CYP1B1 gene. In addition, randomly selected cases with G/G, T/T and G/T variants were sequenced in order to confirm the presence of the A119S change. Sequencing was prepared by using conventional methods.

### Variant 142 G>C (R48G)

The variants of R48G alteration was identified by RFLP-PCR using Eco88I (AvaI) restriction enzyme (Fermentas). PCR was performed with primers e.g. F1CYP (TCCATCCAGCAGACCACGCT) and e.g. R1 (GCCGGACACCACACGGAAG). PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2 (Expand Long Template PCR System Roche—22.5 mM MgCl₂), 200 μM each dATP, dCTP, dGTP i dTTP and 1 U Taq DNA polymerase. In each reaction negative control (control without DNA) was used.

### PCR Conditions:

Initial denaturation −95° C. 5 minutes
29 cycles, each of: denaturation −94° C. 30 s
primer annealing −56° C. 30 s
primer elongation −72° C. 30 s
   elongation −72° C. 5 minutes

Digestion was performed overnight at 37° C. in volume of 24 μl containing: 12 μl PCR product, 10× Buffer Tango (Fermentas) and 2U Eco88I (AvaI) enzyme (Fermentas). Then, 15 μl of digestion product was mixed with 10 μl loading buffer and was electrophoresed in agarose gel (4% agarose gel (SeaKem FMC), IX bufor TBE, 25 μg/ml ethidium bromide) at 6V/cm for 30 minutes. Separated PCR products were visualized in UV light. PCR product (336 bp) was digested on three fragments: 14 bp, 91 bp and 230 bp in cases which containing nucleotide G in 142 nucleotide site of CYP1B1 gene. In addition, randomly selected cases with G/G, C/C and C/G variants were sequenced in order to confirm the presence of the R480 change. Sequencing was prepared by using conventional methods.

### Variant 432 C>G (V432L)

The variants of V432L alteration was identified by RFLP-PCR using OliI restriction enzyme (Fermentas). PCR was performed with primers e.g. CYP1294F (ATGCGCTTCTCCAGCTTTGT) and e.g. CYP1294R (TATGGAGCACACCTCACCTG).

PCR reactions was carried out in PTC—200 Peltier DNA ThermalCycler (MJ Research) in volume of 25 μl included: 1 μl (50 ng) genomic DNA, 4 pmol each primer set, 2.5 μl PCR Buffer 2 (Expand Long Template PCR System Roche—22.5 mM MgCl₂), 200 μM each dATP, dCTP, dGTP i dTTP and 1 U Taq DNA polymerase. In each reaction negative control (control without DNA) was used.

### PCR Conditions:

Initial denaturation −95° C. 15 minutes
10 cycles, each of: denaturation −95° C. 30 s
primer annealing −62-57° C. 30 s
(decrease temperature 0.5° C. in each cycle)
primer elongation −72° C. 2 minutes
   30 cycles, each of: denaturation −95° C. 30 s
primer annealing −57° C. 30 s
primer elongation −72° C. 30 s
   elongation −72° C. 8 minutes

Digestion was performed overnight at 37° C. in volume of 24 μl containing: 12 μl PCR product, 10× Buffer R (Fermentas) and 2U MI enzyme (Fermentas). Then, 15 μl of digestion product was mixed with 10 μl loading buffer and was electrophoresed in agarose gel (3% agarose gel (SeaKem FMC), 1× bufor TBE, 25 μg/ml ethidium bromide) at 6V/cm for 30 minutes. Separated PCR products were visualized in UV light. PCR product (623 bp) was digested on two fragments: 132 bp and 491 bp in cases which containing nucleotide C in 4329 nucleotide site of CYP1B1 gene. In addition, randomly selected cases with G/G, C/C and C/G variants were sequenced in order to confirm the presence of the L432V change. Sequencing was prepared by using conventional methods.

### 7. FGFR2

### Simple Probe Real-Time Polymerase Chain Reaction

### Variant: Rs1219648 MG

Real-Time PCR reactions were carried out in a LightCycler LC-480 Genotyping Master Kit (Roche). The parameters of the PCR reaction were kept exactly as recommended in the protocol of the LightCycler Genotyping Master Kit.

Primers used to detect the variant Rs1219648 A/G were nfgf F 5′ GCG AAT CAT TOG GAC AAG CCA TG 3′ and nfgf R 5′ TCT TCC ATG GTA CCG GTT TC 3′. The corresponding DNA probe is fgfF1pr fam-CAT CCT TGA AGA GCG TGT GTC-pho

### 8. MAP3K1

### Simple Probe Real-Time Polymerase Chain Reaction

### Variant: Rs1219648

Real-Time PCR reactions were carried out in a LightCycler LC-480 Genotyping Master Kit (Roche). The parameters of the PCR reaction were kept exactly as recommended in the protocol of the LightCycler Genotyping Master Kit.

Primers used to detect the variant Rs1219648 A/G were M3kf F 5′CCC ATT ACT TGA GAT GAT CTC TGA G 3′ and M3k R 5′ TAT GGG AAG GAG TCG TTG AG 3′. The corresponding DNA probe is M3kp fam-CTG CTG GAG AAA GGC ATG TGC AA-pho

### 9. P53

### Restriction Fragment Length Polymorphism Polymerase Chain Reaction (RFLP-PCR)

### Variant: R72P

PCR-RFLP analysis of the codon 72 of the TP53 gene originally described by Ara et al. was used to identify TP53 R72P genotypes. The two primers were 5′-CCCGGACGATATTGAACA-3′ and 5′-AGAAGCCCAGACGGAAC-3′. PCR reactions were carried out in PTC—200 Peltier DNA ThermalCycler (MJ Research). Each PCR reaction mixture (50 ml) contained 10 pmol of each primer, 2.0 mM MgCl2, 200 mM each dNTP, 1 unit of Taq polymerase and 100-300 ng of genomic DNA. In each reaction negative control (control without DNA) was used.

### PCR Conditions:

Initial denaturation −94° C. 7 minutes
35 cycles, each of: denaturation −94° C. 30 s
primer annealing −55° C. 60 s
primer elongation −72° C. 60 s
   elongation −72° C. 8 minutes

After confirmation of an amplified fragment of the expected size (199 bp) on an agarose gel, the PCR products were digested with 2 units of restriction enzyme BstUI. (New England Biolabs, Beverly, Mass.) at 60° C. After an overnight digestion, the products were separated by gel electrophoresis (3% agarose gel for 20 minutes at 250 V) and visualized by staining with ethidium bromide. Sequencing was performed by using conventional methods.

### 10. Rs6983267

The Rs6983267 G/T variant was identified by RFLP-PCR using NumCI restriction enzyme (Fermentas). PCR was performed with primers F 5′ CTGAACCTGTGGGTTGGCTGTCA 3′ and R 5′ TAATACCCTCATCGTCCTTTGAG 3′. PCR reactions were carried out in DNA ThermalCycler 9600 (Perkin Elmer). A volume of 15 ul of reaction mixture included: 1 μl (50 ng) genomic DNA genomic DNA, 4 pmol and 6 pmol of each of the primers respectively, 1.3 μl PCR buffer (100 mM Tris-HCl, 500 mM KCL, 15 mM MgCl2, 1 mg/ml gelatin; pH 8.6), 200 μM each dATP, dCTP, dGTP i dTTP and 1 U Taq DNA polymerase. In each reaction negative control (control without DNA) was used.

### PCR Conditions

Initial denaturation −94° C. 10 minutes
35 cycles, each of: denaturation −94° C. 25 s
primer annealing −62° C. 30 s
primer elongation −72° C. 35 s
primer elongation −72° C. 35 s

Digestion was performed overnight at 37° C. in volume of 15 ul containing: 15 ul gel PCR product (197 bp), 1× Red (Fermentas) and NumCI restriction enzyme (Fermentas). Then, 10 ul of digestion product was mixed with 10 ul loading buffer and was electrophoresed in agarose gel (4% agarose gel (SeaKem FMC), 1× buffer TBE, 25 ug/ml ethidium bromide) at 6V/cm for 40 minutes. Separated PCR products were visualized in UV light. PCR product was digested into 3 fragments of lengths 20 bp, 28 bp and 149 bp respectively for the presence of allele T, and alternatively into 2 fragments of length 20 bp, and 177 bp respectively for the presence of allele G.

### 11. TNRC9

### Simple Probe Real-Time Polymerase Chain Reaction

### Variant: Rs1219648 A/G

Real-Time PCR reactions were carried out in a LightCycler LC-480 Genotyping Master Kit (Roche). The parameters of the PCR reaction were kept exactly as recommended in the protocol of the LightCycler Genotyping Master Kit.

Primers used to detect the variant Rs1219648 A/G were Ntnrf F 5′ GCG AAT CAT TGG GAC AAG CCA TG 3′ and Ntnr R 5′ CCT AAT GAT TTT CTC TCC TTA ATC C 3′. The corresponding DNA probe is Ntnr fam-CTC TAT AGC TGT CCC TTA GC-pho.

### 12. XPD

### Restriction Fragment Length Polymorphism Polymerase Chain Reaction (RFLP-PCR)

### Variant D312N

The variants of D312N alteration were identified by RFLP-PCR using Psp 14061 restriction enzyme (Fermentas). PCR was performed with primers e.g. 936gaF (ATCAAAGAGACAGACGAGCAG) and 936gaR (GCTCACCCTGCAGCACAACGT). PCR reactions were carried out in PTC—200 Peltier DNA ThermalCycler (MJ Research). Each PCR reaction mixture (50 ml) contained 10 pmol of each primer, 2.0 mM MgCl2, 200 mM each dNTP, 1 unit of Taq polymerase and 100-300 ng of genomic DNA. In each reaction negative control (control without DNA) was used.

### PCR Conditions:

Initial denaturation −94° C. 7 minutes
35 cycles, each of: denaturation −94° C. 30 s
primer annealing −55° C. 60 s
primer elongation −72° C. 60 s
   elongation −72° C. 8 minutes

After confirmation of an amplified fragment of the expected size on an agarose gel, the PCR products were digested with 2 units of Psp 14061 restriction enzyme (Fermentas) at 60° C. After an overnight digestion, the products were separated by gel electrophoresis (3% agarose gel for 20 minutes at 250 V) and visualized by staining with ethidium bromide. Sequencing was performed by using conventional methods.

### Variant K751Q

The variants of K715Q alteration were identified by RFLP-PCR using PstI restriction enzyme (Fermentas). PCR was performed with 2253F (CTGTTCTCTCCAGGAGGATCAG) and 2253R (GACAGTGAGAAATGTCACCTGAC) primers. PCR reactions were carried out in PTC—200 Peltier DNA ThermalCycler (MJ Research). Each PCR reaction mixture (50 ml) contained 10 pmol of each primer, 2.0 mM MgCl2, 200 mM each dNTP, 1 unit of Taq polymerase and 100-300 ng of genomic DNA. In each reaction negative control (control without DNA) was used.

### PCR Conditions:

Initial denaturation −94° C. 7 minutes
35 cycles, each of: denaturation −94° C. 30 s
primer annealing −55° C. 60 s
primer elongation −72° C. 60 s
   elongation −72° C. 8 minutes

After confirmation of an amplified fragment of the expected size on an agarose gel, the PCR products were digested with 2 units of PstI restriction enzyme (Fermentas) at 60° C. After an overnight digestion, the products were separated by gel electrophoresis (3% agarose gel for 20 minutes at 250 V) and visualized by staining with ethidium bromide. Sequencing was performed by using conventional methods.

### Purification of PCR Products

Sequencing products were placed on Microcon—100 (Amicon) column which fit on 0.5 ml Eppendorf tube. 400 μl distilled water was added, then centrifuged for 15 minutes at 1850 g in 25° C. The columns were 4 times washed with 400 μl of distilled water. After the last washing step, the columns were turned up side down and placed on a new Eppendorf tube. By centrifugation for 3 minutes at 9000 g we obtain 5 μl purified PCR product which were 4 times diluted with distilled water.

TABLE 1
List of studied genes/changes

| Marker | SNP or mutation | reference |
|---|---|---|
| BRCA1 | C61G, 4153delA, 5382insC | 7, 8 |
| CHEK2 | IVS + 1G/A, I157T, 1100delC, | 9, 10 |
| | del5395 | |
| p53 | R72P | 11 |
| XPD-GG | D312N | 12 |
| XPD-AA | K751Q | 12 |
| XPD-CC/AA | K751Q/D312N | 12 |
| CYP1B1 | R48G, A119S, V432L: | |
| | haplotype GG⁴⁸TT¹¹⁹CC⁴³² | 13 |
| NOD2/CARD15 | 3020insC | 5, 14 |
| BRCA2 | T1915M | 15 |
| CDKN2A (p16) | A148T | 16 |
| FGFR2-nAA | Rs1219648, AA excluded | 17 |
| FGFR2-GG | Rs1219648, only GG | 17 |
| TNRC9-nTT | Rs3803662, TT excluded | 1, 18 |
| MAP3K1-nAA | Rs889312, AA excluded | 1 |
| Rs6983267 | Rs69S3267, only GG | 19 |
| FGFR2-nAA and | Combined genotypes of | 1 |
| TNRC9-nTT | FGFR2 and TNRC9 | |

Genetic changes selected for analysis included alterations which have previously been shown to be associated with an increased risk of consecutive unselected breast cancers or of their sub-types.

### Data Analysis

In the first stage. BRCA1 mutation carriers were excluded. In order to find the other most optimal panels of markers, all samples were analyzed and then the marker selected based on the highest odds ration (OR) value in a comparison between cases and controls using Fischer's exact test. After the selection of markers all samples matching it were removed and then the process reiterated until the markers with OR>1 and occurring in less than 90% of controls could be found.

### Results

Statistically significant differences between cases and controls have been found for 19 of 20 analyzed groups (Tab. 2-4) except of one small group (n=63) of ductal, high grade early onset cancers.

TABLE 2
Frequency of identified panel of markers
in all consecutive cancers and controls

| Gene/Marker | Cases (%) | | Controls (%) | |
|---|---|---|---|---|
| BRCA1 | 2.7% | (26/977) | 0% | (0/977) |
| CHEK2 | 11.9% | (113/951) | 6% | (59/977) |
| P53 | 10.1% | (85/838) | 5.7% | (52/918) |
| TNRC9 | 55.6% | (419/753) | 45.8% | (397/866) |
| FGFR2 - GG | 18.3% | (61/334) | 13.9% | (65/469) |
| CDKN2A | 7% | (19/273) | 5.4% | (22/404) |
| XPD - GG | 41% | (104/254) | 36.4% | (139/382) |
| XPD - CC/AA | 17.3% | (26/150) | 14% | (34/243) |
| BRCA2 | 7.3% | (9/124) | 4.8% | (10/209) |
| XPD - AA | 20% | (23/115) | 18.6% | (37/199) |
| Any marker | 90.6% | (885/977) | 83.4% | (815/977) |

| | |
|---|---|
| Statistical significance | P = 3 · 10⁻⁶ |

Genetic changes were present in more than 90% of breast cancer patients in 18 of 20 groups except of ductal cancers ER (−) (Tab. 4). The highest proportion of cases with constitutional changes—99.3% (139/140) was observed for lobular cancers (Tab. 3). List of genetic markers and/or the level of their association with cancer predisposition reflected by their position on the list was different between groups. No single marker was identified as being associated with cancer in all groups except of BRCA1 mutations included on all lists by definition.

TABLE 3
Frequency of identified panel of markers in
a group of lobular cancers and in controls

| Gene/Marker | Cases (%) | | Controls (%) | |
|---|---|---|---|---|
| BRCA1 | 0.7% | (1/140) | 0% | (0/140) |
| CHEK2 | 19.4% | (27/139) | 4.3% | (6/140) |
| P53 | 10.7% | (12/112) | 6% | (8/134) |
| BRCA2 | 9% | (9/100) | 4.8% | (9/126) |
| FGFR2 - nAA | 75.8% | (69/91) | 60% | (72/120) |
| TNRC9 | 72.7% | (16/22) | 39.6% | (19/48) |
| NOD2/CARD15 | 16.7% | (1/6) | 0% | (0/29) |
| MAP3K1 - nAA | 80% | (4/5) | 41.4% | (12/29) |
| Any marker | 99.30% | (139/140) | 87.90% | ( )123/140 |

| | |
|---|---|
| Statistical significance | P = 0.00073 |

TABLE 4
Frequency of identified panels of markers
in different groups of breast cancers

| Group | Cases (%) | | Controls (%) | | p |
|---|---|---|---|---|---|
| Consecutive | 90.6% | (885/977) | 83.4% | (815/977) | 3 · 10⁻⁶ |
| Dgn < 51 | 91.0% | (282/310) | 81.9% | (254/310) | 0.0014 |
| Dgn > 50 | 96.1% | (641/667) | 89.4% | (596/667) | 1 · 10−8 |
| Ductal, low | 94.8% | | 89.1% | | 0.0041 |
| grade | | | | | |
| Ductal, high | 95.2% | | 83.8% | | 0.0010 |
| grade | | | | | |
| Lobular | 99.30% | | 87.90% | | 0.00073 |
| ER (+) | 96.3% | | 90.0% | | 2.4 · 10−5 |
| ER (−) | 93.5% | | 80.1% | | 9.6 · 10−5 |
| Ductal, low | 96.2% | | 88.0% | | 0.0006 |
| grade, dgn < 51 | | | | | |
| Ductal, high | 96.2% | (100/104) | 86.5% | (91/104) | 0.0401 |
| grade, dgn > 50 | | | | | |
| ER (+), dgn > 50 | 94.6% | (316/334) | 86.2% | (288/334) | 0.0003 |
| ER (−), dgn > 50 | 96.6% | (114/118) | 82.2% | (97/118) | 0.0005 |
| Ductal, low | 94.1% | | 80.7% | | 0.0015 |
| grade, dgn < 50 | | | | | |
| Ductal, high | 96.8% | (61/63) | 88.9% | (56/63) | 0.1638 |
| grade, dgn < 51 | | | | | |
| ER (+), dgn < 51 | 96.0% | (167/174) | 87.9% | (153/174) | 0.0097 |
| ER (−), dgn < 51 | 92.8% | (77/83) | 78.3% | (65/83) | 0.0139 |
| Ductal, low | 96.1% | | 90.0% | | 0.0087 |
| grade, ER (+) | | | | | |
| Ductal, low | 81.8% | | 58.2% | | 0.0119 |
| grade, EH (−) | | | | | |
| Ductal, high | 98.7% | (75/76) | 84.2% | (64/76) | 0.0023 |
| grade, ER (+) | | | | | |
| Ductal, high | 84.9% | (62/73) | 61.6% | (45/73) | 0.0025 |
| grade, ER (−) | | | | | |

Markers associated with majority of groups include CHEK2, p53, TNRC9nTT, FGFR2nAA, XPD CC/AA and XPD GG. Some markers were tightly associated with particular groups of patients for example CDKN2A with ductal cancers diagnosed under age of 51 years that were high grade and ER (+), CYP1B1 with ductal cancers of low grade and diagnosed under age of 51 years of age, MAP3K1 nAA with cancers diagnosed over age of 50 years and ER (−), Rs6983267 with ductal cancers of high grade and diagnosed above the age of 50 yrs.

Tables 5 to 22 show further panels of marker combinations characteristic for a significantly decreased risk of developing cancer or a particular cancer subtype (cancer site, cancer grade and/or estrogen-receptor status) in different subgroups of patients (divided by age of diagnosis).

TABLE 5
Frequency of identified panel of markers in a group of
cancers diagnosed under the age of 51 and in controls

| Gene/Marker | Cases (%) | | Controls (%) | |
|---|---|---|---|---|
| BRCA1 | 3.9% | (12/310) | 0% | (0/310) |
| CKEK 2 | 13.8% | (41/298) | 8.7% | (27/310) |
| CDKN2A | 5.8% | (15/257) | 3.8% | (11/283) |
| p 53 | 8.3% | (20/242) | 6.3% | (17/272) |
| FGFR - nAA | 62.6% | (139/222) | 57.3% | (146/255) |
| XPD - CC/AA | 12% | (10/83) | 5.5% | (6/109) |
| XPD - GG | 47.9% | (35/73) | 33.9% | (35/103) |
| RS 67 | 26.3% | (10/38) | 17.6% | (12/68) |
| Any marker | 91.0% | (282/310) | 81.9% | (254/310) |

| | |
|---|---|
| Statistical significance | p = 0.0014 |

TABLE 6
Frequency of identified panel of markers in a group of
cancers diagnosed above the age of 50 and in controls

| Gene/Marker | Cases (%) | | Controls (%) | |
|---|---|---|---|---|
| BRCA1 | 2.1% | (14/667) | 0% | (0/667) |
| CHEK 2 | 11% | (72/653) | 4.8% | (32/667) |
| p 53 | 11% | (64/581) | 5.4% | (34/635) |
| TNR | 55.9% | (289/517) | 45.3% | (272/601) |
| FGFR - nAA | 68.9% | (157/228) | 60.8% | (200/329) |
| BHCA 2 | 5.6% | (4/71) | 2.3% | (3/129) |
| XPD - CC/AA | 13.4% | (9/67) | 6.3% | (8/126) |
| NOD 2 | 13.8% | (8/58) | 6.8% | (8/118) |
| XPD - GG | 48% | (24/50) | 35.5% | (39/110) |
| Any marker | 96.1% | (641/667) | 89.4% | (596/667) |

| | |
|---|---|
| Statistical significance | P = 2.4 · 10⁻⁶ |

TABLE 7
Frequency of identified panel of markers in a group
of ductal cancers, low grade and in controls

| Gene/Marker | Cases (%) | | Controls (%) | |
|---|---|---|---|---|
| BRCA1 | 1% | (4/401) | 0% | (0/401) |
| p 53 | 10.1% | (40/397) | 5.7% | (23/401) |
| CYP1B1 | 8.4% | (30/357) | 4.8% | (18/378) |
| CHEK 2 | 10.4% | (34/327) | 6.7% | (24/360) |
| TNR | 56% | (164/293) | 44.3% | (149/336) |
| XPD - GG | 41.1% | (53/129) | 34.2% | (64/187) |
| XPD - AA | 18.4% | (14/76) | 12.2% | (15/123) |
| FGFR - nAA | 66.1% | (41/62) | 59.3% | (64/108) |
| Any marker | 94.8% | (380/401) | 89.1% | (357/401) |

| | |
|---|---|
| Statistical significance | p = 0.0041 |

TABLE 8
Frequency of identified panel of markers in a group
of ductal cancers, high grade and in controls

| Gene/Marker | Cases (%) | | Controls (%) | |
|---|---|---|---|---|
| BRCA1 | 7.2% | (12/167) | 0% | (0/167) |
| XPD - CC/AA | 12.3% | (19/155) | 6.6% | (11/167) |
| TNR | 61.0% | (83/136) | 43.6% | (68/156) |
| CHEK 2 | 11.3% | (6./53) | 5.1% | (4/79) |
| CDKN2A | 14.9% | (7/47) | 6.7% | (5/75) |
| p 53 | 15.0% | (6/40) | 10.0% | (7/70) |
| RS 67 | 26.4% | (9/34) | 19.0% | (12/63) |
| FGFR - GG | 12.0% | (3/25) | 5.9% | (3/51) |
| XPD - GG | 54.5% | (12/22) | 41.7% | (20/48) |
| NOD2 | 20.0% | (2/10) | 3.6% | (1/28) |
| Any marker | 95.2% | (159/167) | 83.8% | (140/167) |

| | |
|---|---|
| Statistical significance | p = 0.0010 |

TABLE 9
Frequency of identified panel of markers in
a group of cancers ER(+) and in controls

| Gene/Marker | Cases (%) | | Controls (%) | |
|---|---|---|---|---|
| BRCA1 | 1.2% | (6/508) | 0% | (0/508) |
| CHEK 2 | 12.7% | (64/502) | 5.9% | (30/508) |
| p 53 | 11.6% | (51/438) | 6.3% | (30/478) |
| TNR | 59.2% | (229/387) | 44.2% | (198/448) |
| CDKN2A | 7.0% | (11/158) | 4.4% | (11/250) |
| BRCA2 | 8.2% | (12/147) | 5.0% | (12/239) |
| FGFR - nAA | 68.1% | (92/135) | 58.1% | (132/227) |
| CYP1B1 | 16.3% | (7/43) | 9.5% | (9/95) |
| XPD - CC/AA | 8.3% | (3/36) | 5.8% | (5/86) |
| XPD - GG | 42.4% | (14/33) | 32.1% | (26/81) |
| M3K - CC | 10.5% | (2/19) | 7.3% | (4/55) |
| Any marker | 96.3% | (491/508) | 90.0% | (457/508) |

| | |
|---|---|
| Statistical significance | P = 2.4 · 10⁻⁵ |

TABLE 10
Frequency of identified panel of markers in
a group of cancers ER(−) and in controls

| Gene/Marker | Cases (%) | | Controls (%) | |
|---|---|---|---|---|
| BRCA1 | 7.0% | (14/201) | 0% | (0/201) |
| CDKN2A | 5.0% | (11/187) | 3.0% | (6/201) |
| XPD - CC/AA | 14.2% | (25/176) | 7.7% | (15/195) |
| p 53 | 9.9% | (15/151) | 5.0% | (9/180) |
| M3K - nAA | 44.9% | (61/136) | 33.3% | (57/171) |
| XPD - AA | 52.0% | (39/75) | 41.2% | (47/114) |
| RS 67 | 30.6% | (11/36) | 14.9% | (10/67) |
| FGFR - nAA + TNR - nTT | 40.0% | (10/25) | 19.3% | (11.57) |
| CYP1B1 | 13.3% | (2/15) | 13.0% | (6/46) |
| Any marker | 93.5% | (188/201) | 80.1% | (161/201) |

| | |
|---|---|
| Statistical significance | P = 9.6 · 10⁻⁵ |

TABLE 11
Frequency of identified panel of markers in a group of ductal
cancers, low grade diagnosed at age above 50 and in controls

| Gene/Marker | Cases (%) | | Controls (%) | |
|---|---|---|---|---|
| BRCA1 | 1.5% | (4/266) | 0% | (0/266) |
| p 53 | 11.8% | (31/262) | 5.3% | (14/266) |
| FGFR - nAA | 72.3% | (167/231) | 59.9% | (151/252) |
| CHEK 2 | 9.4% | (6/64) | 5.0% | (5/101) |
| XPD - AA | 44.8% | (26/58) | 32.3% | (31/96) |
| XPD - GG | 31.3% | (10/32) | 12.3% | (8/65) |
| TNR - nTT | 54.5% | (12/22) | 43.9% | (25/57) |
| Any marker | 96.2% | (256/266) | 88.0% | (239/266) |

| | |
|---|---|
| Statistical significance | p = 0.0006 |

TABLE 12
Frequency of identified panel of markers in a group of ductal
cancers, high grade diagnosed at age above 50 and in controls

| Gene/Marker | Cases (%) | | Controls (%) | |
|---|---|---|---|---|
| BRCA1 | 4.8% | (5/104) | 0% | (0/104) |
| TNR - n TT | 58.6% | (58/99) | 41.3% | (43/104) |
| XPD - CC/AA | 22.0% | (9/41) | 9.8% | (6/61) |
| RS 67 | 21.9% | (7/32) | 12.7% | (7/55) |
| p 53 | 16.0% | (4/25) | 8.3% | (4/48) |
| NOD2 | 19.0% | (4/21) | 9.1% | (4/44) |
| CHEK2 | 17.6% | (3/17 | 10.0% | (4/40) |
| M3K - nAA | 42.9% | (6/14 | 36.1% | (13/36) |
| CDKN2A | 12.5% | (1/8) | 4.3% | (1/23) |
| XPD - GG | 42.9% | (3/7) | 40.9 | (9/22) |
| Any marker | 96.2% | (100/104) | 86.5% | (91/104) |

| | |
|---|---|
| Statistical significance | p = 0.0401 |

TABLE 13
Frequency of identified panel of markers in a group of
cancers ER (+) diagnosed at age above 50 and in controls

| Gene/Marker | Cases (%) | | Controls (%) | |
|---|---|---|---|---|
| BRCA1 | 0.9% | (3/334) | 0% | (0/334) |
| CHEK2 | 11.8% | (39/331) | 4.8% | (16/334) |
| TNR - nTT | 57.5% | (168/292) | 43.1% | (137/318) |
| p 53 | 15.3% | (19/124) | 6.6% | (12/181) |
| FGFR - nAA | 73.3% | (77/105) | 60.4% | (102/169) |
| CYB1B1 | 10.7% | (3/28) | 9% | (6/67) |
| XPD - AA | 28.0% | (7/25) | 24.6% | (15/61) |
| Any marker | 94.6% | (316/334) | 86.2% | (288/334) |

| | |
|---|---|
| Statistical significance | p = 0.0003 |

TABLE 14
Frequency of identified panel of markers in a group of
cancers ER (−) diagnosed at age above 50 and in controls

| Gene/Marker | Cases (%) | | Controls (%) | |
|---|---|---|---|---|
| BRCA1 | 5.9% | (7/118) | 0% | (0/118) |
| XPD - CC/AA | 14.4% | (16/111) | 5.9% | (7/118) |
| M3K - nAA | 47.4% | (45/95) | 28.8% | (32/111) |
| CDKN2A | 12% | (6/50) | 1.6% | (1/79) |
| FGFR - nAA + TNR - nTT | 31.8% | (14/44) | 16.7% | (13/78) |
| RS 67 | 33.3% | (10/30) | 20% | (13/65) |
| XPD - AA | 65% | (13/20) | 40.4% | (21/52) |
| XPD - GG | 14.3% | (1/7) | 6.5% | (2/31) |
| CYP1B1 | 20% | (1/5) | 16% | (4/25) |
| Any marker | 96.6% | (114/118) | 82.2% | (97/118) |

| | |
|---|---|
| Statistical significance | p = 0.0005 |

TABLE 15
Frequency of identified panel of markers in a group of ductal
cancers, low grade diagnosed at age under 51 and in controls

| Gene/Marker | Cases (%) | | Controls (%) | |
|---|---|---|---|---|
| BRCA1 | 0% | (0/135) | 0% | (0/135) |
| FGFR - GG | 18.5% | (25/135) | 10.4% | (14/135) |
| CYP1B1 | 10.0% | (11/110) | 5.8% | (7/121) |
| TNR - nTT | 59.6% | (59/99) | 44.7% | (51/114) |
| RS 67 | 27.5% | (11/40) | 14.3% | (9/63) |
| p 53 | 10.3% | (3/29) | 5.6% | (3/54) |
| XPD - AA | 46.2% | (12/26) | 33.3% | (17/51) |
| BRCA2 | 21.4% | (3/14) | 2.9% | (1/34) |
| XPD - CC/AA | 18.2% | (2/11) | 15.2% | (5/33) |
| CHEK2 | 11.1% | (1/9) | 7.1% | (2/28) |
| Any marker | 94.1% | (127/135) | 80.7% | (109/135) |

| | |
|---|---|
| Statistical significance | p = 0.0015 |

TABLE 16
Frequency of identified panel of markers in a group of ductal
cancers, high grade diagnosed at age under 51 and in controls

| | | |
|---|---|---|
| Gene/Marker | Cases (%) | Controls (%) |

| | | | | |
|---|---|---|---|---|
| BRCA1 | 11.1% | (7/63) | 0% | (0/63) |
| CDKN2A | 14.3% | (8/56) | 4.8% | (3/63) |
| XPD - CC/AA | 12.5% | (6/48) | 5.0% | (3/60) |
| p 53 | 16.7% | (7/42) | 5.3% | (3/57) |
| XPD - GG | 51.4% | (18/35) | 35.2% | (19/54) |
| NOD2 | 17.% | (3/17) | 0% | (0/35) |
| TNR - n TT | 64.3% | (9/14) | 62.8% | (22/35) |
| FGFR - nAA | 60.0% | (3/5) | 46.2% | (6/13) |
| Any marker | 96.8% | (61/63) | 88.9% | (56/63) |

| | |
|---|---|
| Statistical significance | p = 0.1638 |

TABLE 17
Frequency of identified panel of markers in a group of breast
cancers ER (+) diagnosed at age under 51 and controls

| Gene/Marker | Cases (%) | | Controls (%) | |
|---|---|---|---|---|
| BRCA1 | 1.7% | (3/174) | 0% | (0/174) |
| CDKN2A | 5.8% | (10/171) | 3.4% | (6/174) |
| TNR - nTT | 61.5% | (99/161) | 47.6% | (80/168) |
| p 53 | 12.9% | (8/62) | 5.7% | (5/88) |
| BRCA2 | 11.1% | (6/54) | 6% | (5/83) |
| XPD - GG | 47.9% | (23/48) | 38.5% | (30/78) |
| FGFR - nAA | 64% | (16/25) | 54.2% | (26/48) |
| XPD - CC/AA | 22.2% | (2/9) | 4.5% | (1/22) |
| Any marker | 96.0% | (167/174) | 87.9% | (153/174) |

| | |
|---|---|
| Statistical significance | p = 0.0097 |

TABLE 18
Frequency of identified panel of markers in a group of
cancers ER (−) diagnosed at age under 51 and in controls

| | | |
|---|---|---|
| Gene/Marker | Cases (%) | Controls (%) |

| | | | | |
|---|---|---|---|---|
| BRCA1 | 8.4% | (7/83) | 0% | (0/83) |
| p 53 | 11.8% | (9/76) | 7.2% | (6/83) |
| TNR - nTT | 47.8% | (32/67) | 51.9% | (40/77) |
| RS 67 | 31.4% | (11/35) | 13.5% | (5/37) |
| FGFR - GG | 20.8% | (5/24) | 3.1% | (1/32) |
| CHEK 2 | 15.8% | (3/19) | 3.2% | (1/31) |
| XPD - GG | 62.5% | (10/16) | 40% | (12/30) |
| Any marker | 92.8% | (77/83) | 78.3% | (65/83) |

| | |
|---|---|
| Statistical significance | p = 0.0139 |

TABLE 19
Frequency of identified panel of markers in a group
of ductal cancers, low grade, ER (+) and in controls

| Gene/Marker | Cases (%) | | Controls (%) | |
|---|---|---|---|---|
| BRCA1 | 0.8% | (2/259) | 0% | (0/259) |
| p 53 | 11.7% | (30/257) | 5.4% | (14/259) |
| TNR - nTT | 57.3% | (130/227) | 43.7% | (107/245) |
| CYP1B1 | 11.3% | (11/97) | 6.5% | (9/138) |
| FGFR - nAA | 67.4% | (58/86) | 60.5% | (78/129) |
| CHEK2 | 7.1% | (2/28) | 5.9% | (3/51) |
| XPD - AA | 42.3% | (11/26) | 31.3% | (15/48) |
| XPD - GG | 26.7% | (4/15) | 15.2% | (5/33) |
| BRCA2 | 9.1% | (1/11) | 7.1% | (2/28) |
| Any marker | 96.1% | (249/259) | 90.0% | (233/259) |

| | |
|---|---|
| Statistical significance | p = 0.0087 |

TABLE 20
Frequency of identified panel of markers in a group
of ductal cancers, low grade, ER (−) and in controls

| | | |
|---|---|---|
| Gene/Marker | Cases (%) | Controls (%) |

| | | | | |
|---|---|---|---|---|
| BRCA1 | 1.8% | (1/55) | 0% | (0/55) |
| CYP1B1 | 11.1% | (6/54) | 1.8% | (1/55) |
| XPD - CC/AA | 12.5% | (6/48) | 5.6% | (3/54) |
| TNR - nTT | 50.0% | (21/42) | 37.3% | (19/51) |
| RS 67 | 42.9% | (9/21) | 18.8% | (6/32) |
| CHEK2 | 8.3% | (1/12) | 3.8% | (1/26) |
| NOD2 | 9.1% | (1/11) | 8.0% | (2/25) |
| Any marker | 81.8% | (45/55) | 58.2% | (32/55) |

| | |
|---|---|
| Statistical significance | p = 0.0119 |

TABLE 21
Frequency of identified panel of markers in a group
of ductal cancers, high grade, ER (+) and in controls

| | | |
|---|---|---|
| Gene/Marker | Cases (%) | Controls (%) |

| | | | | |
|---|---|---|---|---|
| BRCA1 | 3.9% | (3/76) | 0% | (0/76) |
| TNR - nTT | 61.6% | (45/73) | 42.1% | (32/76) |
| XPD - CC/AA | 21.4% | (6/28) | 6.8% | (3/44) |
| CYP1B1 | 18.2% | (4/22) | 4.9% | (2/41) |
| CHEK 2 | 11.1% | (2/18) | 2.6% | (1/39) |
| CDKN2A | 31.3% | (5/16) | 10.6% | (4/38) |
| FGFR - nAA | 81.8% | (9/11) | 55.9% | (19/34) |
| p 53 | 50.0% | (1/2) | 23.3% | (3/15) |
| Any marker | 98.7% | (75/76) | 84.2% | (64/76) |

| | |
|---|---|
| Statistical significance | p = 0.0023 |

TABLE 22
Frequency of identified panel of markers in a group
of ductal cancers, high grade, ER (−) and in controls

| | | |
|---|---|---|
| Gene/Marker | Cases (%) | Controls (%) |

| | | | | |
|---|---|---|---|---|
| BRCA1 | 9.6% | (7/73) | 0% | (0/73) |
| p 53 | 12.1% | (8/66) | 2.7% | (2/73) |
| XPD - CC/AA | 13.8% | (8/58) | 4.2% | (3/71) |
| NOD 2 | 14.0% | (7/50) | 5.9% | (4/68) |
| FGFR - GG | 16.3% | (7/43) | 9.4% | 6/64) |
| RS 67 | 25.0% | (9/36) | 12.1% | (7/58) |
| M3K - nAA | 59.3% | (16/27) | 45.1% | (23/51) |
| Any marker | 84.9% | (62/73) | 61.6% | (45/73) |

| | |
|---|---|
| Statistical significance | p = 0.0025 |

### CONCLUSIONS

Evidence is presented of there being a series of low penetrant genetic polymorphisms that account for a proportion of disease risk observed in a series of breast cancer sub-types derived from the homogeneous Polish population. The data presented for breast cancer suggests that the approach utilized in this study could be applicable for the identification of low penetrance genetic risk factors in other malignancies.

The results presented appear not to reflect any major bias as the statistical significance clearly differentiates cases from controls in 19 of the 20 groups. Furthermore the selected genetic markers are present in more than 90% of cases in 18 of the 20 subgroups examined. Additionally, for all groups with at least 100 cases, patient/control pairs were divided randomly into two sub-groups with all results remaining consistent (data not shown). Additionally, the degree of accuracy of the registry used for this analysis belongs to one of the largest reported to date (>95% of consecutive cases, cancer free controls).

The most significant results have been obtained for lobular cancers for which the genetic markers were represented in more than 99% of the cases. This may be related to the greater contribution of genetic factors in the development of this sub-type of breast cancer and, therefore, reflects a larger amount of constitutional change associated with this type of disease. For many years it has been suggested that the increased frequency of multifocal and bilateral disease are characteristic features of lobular cancer, which is consistent with features that are characteristic of breast cancers derived from patients with strong family histories of disease 20-22).

The data also demonstrates how much can be achieved if cancers are accurately stratified by clinical characteristics and/or pathology. Certainly, some genetic polymorphisms that are critical for the development of certain sub-types of cancer cannot be identified if association studies are limited to the analysis of consecutive unselected cases.

The results achieved in this study suggest that there are two major groups of genes: Those that can be identified by analyses of consecutive unselected cases and represent a more general genetic risk factor; and those that are more specific in nature since they appear to be determining factors for sub-types of disease which can only be identified after appropriate stratification.

The data do not exclude the role of the environmental factors but suggest that they act effectively on predisposed persons. The probability of being affected by cancer without any constitutional changes associated with disease predisposition seems to be extremely low. For example—only 3.9% of women with breast cancer diagnosed above the age of 50 years do not carry any of identifiable markers used in this study, it may be possible to categorize women aged above 50 years without any markers as having a breast cancer risk of more than 25 times less than someone with one or more markers.

The results of this study suggest that after further improvements of testing with the use of low/moderate genetic cancer risk markers, DNA analysis will be the initial starting point for prevention, surveillance and treatment schemes for all adults.

### REFERENCES

1. Easton D F, Pooley K A, Dunning A M, Pharoah P D, Thompson D, Ballinger D G, Struewing J P, Morrison J, Field H, Luben R, Wareham N, Ahmed S, Healey C S et al (2007) Genome-wide association study identifies novel breast cancer susceptibility loci. Nature 28; 447(7148):1087-93
2. The Wellcome Case Control Consortium (2007) Genome-wide association study of 14,000 cases of seven common diseases and 3000 shared controls. Nature 447:661-78
3. Debniak T, Cybulski C, Gorski B, Huzarski T, Byrski T, Gronwald J, Jakubowska A, Kowalska E, Oszurek O, Narod S A, Lubinski J (2007) CDKN2A-positive breast cancers in young women from Poland. Breast Cancer Res Treat 103(3): 355-9
4. Cybulski C, Gorski B, Huzarski T, Byrski T, Gronwald J, Debniak T, Wokolorczyk D, Jakubowska A, Kowalska E, Oszurek O, Narod S A, Lubinski J (2006) CHEK2-positive breast cancers in young Polish women. Clin Cancer Res 12(16):4832-5
5. Huzarski T, Lener M, Domagala W, Gronwald J, Byrski T, Kurzawski G, Suchy J, Chosia M, Woyton J, Ucinski M, Narod S A, Lubinski J (2005) The 3020insC allele of NOD2 predisposes to early-onset breast cancer. Breast Cancer Res Treat 89(1):91-3
6. Huijts P E, Vreeswijk M P, Kroeze-Jansema K H, Jacobi C E, Seynaeve C, Krol-Warmerdam E M, Wijers-Koster P M, Blom J C, Pooley K A, Klijn J G, Tollenaar R A, Devilee P, van Asperen C J (2007) Clinical correlates of low-risk variants in FGFR2, TNRC9, MAP3K1, LSP1 and 8q24 in a Dutch cohort of incident breast cancer cases. Breast Cancer Res 9(6):R78 [Epub]
7. Gorski B., Byrski T., Huzarski T., Jakubowska A., Menkiszak J., Gronwald J., Pluzanska A., Bebenek M., Fischer-Maliszewska L., Grzybowska E., Narod S. A., Lubinski J (2000) Founder Mutations in the BRCA1 Gene in Polish Families with Breast-Ovarian Cancer. Am J Hum Genet 66:1963-1968
8. Gorski B, Jakubowska A, Huzarski T, Byrski T, Gronwald J, Grzybowska E, Mackiewicz A, Stawicka M, Bebenek M, Sorokin D, Fiszer-Maliszewska L, Haus O, Janiszewska H, Niepsuj S, Gozdz S, Zaremba L, Posmyk M, Pluzanska M, Kilar E, Czudowska D, Wasko B, Miturski R, Kowalczyk J R, Urbanski K, Szwiec M, Koc J, Debniak B, Rozmiarek A, Debniak T, Cybulski C, Kowalska E, Toloczko-Grabarek A, Zajaczek S, Menkiszak J, Medrek K, Masojc B, Mierzejewski M, Narod S A, Lubinski J (2004) A high proportion of founder BRCA1 mutations in Polish breast cancer families. Int J Cancer 110(5):683-686
9. Cybulski C, Gorski B, Huzarski T, Masojc B, Mierzejewski M, Debniak T, Teodorczyk U, Byrski T, Gronwald J, Matyjasik J, Zlowocka E, Lenner M, Grabowska E, Nej K, Castaneda J, Medrek K, Szymanska A, Szymanska J, Kurzawski G, Suchy J, Oszurek O, Witek A, Narod S A, Lubinski J (2004) CHEK2 Is a Multiorgan Cancer Susceptibility Gene. Am J Hum Genet 75(6):1131-1135
10. Cybulski C, Wokolorczyk D, Huzarski T, Byrski T, Gronwald J, Gorski B, Debniak T, Masojc B. Jakubowska A, Gliniewicz B, Sikorski A, Stawicka M, Godlewski D, Kwias Z, Antczak A, Krajka K, Lauer W, Sosnowski M, Sikorska-Radek P, Bar K, Klijer R, Zdrojowy R, Malkiewicz B, Borkowski A, Borkowski T, Szwiec M, Narod S A, Lubinski J (2006) A large germline deletion in the CHEK2 kinase gene is associated with an increased risk of prostate cancer. J Med Genet 43(11):863-866
11. Medrek K, Gorski B, Chudecka-Glaz A, Magnowski P, Jakubowska A, Debniak T, Cybulski C, Masojc B, Grunwald J, Huzarski T, Byrski T, Matyjasik J, Zlowocka E, Oszutowska A, Nej-Wolosiak K, Klany J, Jaworowska E, Rzepka-Gorska I, Spaczynski M, Narod S A, Lubinski J (2007) The 215G>C polymorphism of p53 is associated with well-differentiated breast and ovarian cancers. Cancer Epidemiol Biomarkers Prey (submitted)
12. Debniak T, Scott R J, Huzarski T, Byrski T, Masojc B, van de Wetering T, Serrano-Fernandez P, Gorski B, Cybulski C, Gronwald J, Debniak B, Maleszka R, Kladny J, Bieniek A, Nagay L, Haus O, Grzybowska E, Wandzel P, Niepsuj S, Narod S A, Lubinski J (2006) XPD common variants and their association with melanoma and breast cancer risk. Breast Cancer Res Treat 98(2):209-15
13. Matyjasik J, Cybulski C, Masojc B, Jakubowska A, Serrano-Fernandez P, Gorski B, Debniak T, Huzarski T, Byrski T, Gronwald J, Zlowocka E, Narod S A, Scott R, Lubinski J (2007) CYP1B1 and predisposition to breast cancer in Poland. Breast Cancer Res Treat 106(3):383-8
14. Lubinski J, Huzarski T, Kurzawski G, Suchy J, Masojc B, Mierzejewski M, Lener M, Domagala W, Chosia M, Teodorczyk U, Medrek K, Debniak T, Zlowocka E, Gronwald J, Byrski T, Grabowska E, Nej K, Szymanska A, Szymanska J, Matyjasik J, Cybulski C, Jakubowska A, Gorski B, Narod S A (2005) The 3020insC Allele of NOD2 Predisposes to Cancers of Multiple Organs. Her Can in Clin Pract 3(2):59-63
15. Gorski B, Narod S A, Lubinski J (2005) A common missense variant in BRCA2 predisposes to early onset breast cancer. Breast Cancer Res 7(6):R1023-7.
16. Debniak T, Gorski B, Huzarski T, Byrski T, Cybulski C, Mackiewicz A, Gozdecka-Grodecka S, Gronwald J, Kowalska E, Haus O, Grzybowska E, Stawicka M, Szwiec M, Urbanski K, Niepsuj S, Wasko B, Gozdz S, Wandzel P, Szczylik C, Surdyka D, Rozmiarek A, Zambrano O, Posmyk M, Narod S A, Lubinski J (2005) A common variant of CDKN2A (p16) predisposes to breast cancer. J Med Genet 42(10):763-5
17. Hunter D J, Kraft P, Jacobs K B, Cox D G, Yeager M, Hankinson S E, Wacholder S, Wang Z, Welch R, Hutchinson A, Wang J, Yu K, Chatterjee N, Orr N, Willett W C, Colditz G A, Ziegler R G, Berg C D, Buys SS, McCarty C A, Feigelson H S, Calle E E, Thun M J, Hayes R B, Tucker M, Gerhard D S, Fraumeni J F Jr, Hoover R N, Thomas G, Chanock S J (2007) A genome-wide association study identifies alleles in FGFR2 associated with risk of sporadic postmenopausal breast cancer. Nat Genet 39(7):870-4
18. Stacey S N, Manolescu A, Sulem P, Rafnar T, Gudmundsson J, Gudjonsson S A, Masson G, Jakobsdottir M, Thorlacius S, Helgason A, Aben K K, Strobbe U, Albers-Akkers M T, Swinkels D W, Henderson B E, Kolonel L N, Le Marchand L, Millastre E, Andres R, Godino J, Garcia-Prats M D, Polo E, Tres A, Mouy M, Saemundsdottir J, Backman V M, Gudmundsson L, Kristjansson K, Bergthorsson J T, Kostic J, Frigge M L, Geller F, Gudbjartsson D, Sigurdsson H, Jonsdottir T, Hrafnkelsson J, Johannsson J, Sveinsson T, Myrdal G, Grimsson H N, Jonsson T, von Hoist S, Werelius B, Margolin S, Lindblom A, Mayordomo J I, Haiman C A, Kiemeney L A, Johannsson O T, Gulcher J R, Thorsteinsdottir U, Kong A, Stefansson K (2007) Common variants on chromosomes 2q35 and 16q12 confer susceptibility to estrogen receptor-positive breast cancer. Nat Genet 39(7):865-9
19. Wokolorczyk D, Gliniewicz B, Sikorski A, Serrano-Fernandez P, Zlowocka E, Masojc B, Debniak T, Matyjasik J, Mierzejewski M, Medrek K, Oszutowska D, Gronwald J, Huzarski T, Byrski T, Jakubowska A, Gorski B, Cybulski C, Narod S A, Lubinski J (2008) A wide range of cancers is associated with the rs6983267 marker on chromosome 8. Cancer Research (submitted)
20. Broet P, de la Rochefordiere A, Scholl S M, Fourquet A, Mosseri V, Durand J C, Pouillart P, Asselain B (1995) Contralateral breast cancer: annual incidence and risk parameters. J Clin Oncol 13(7):1578-83
21. Lishman S C, Lakhani S R (1999) Atypical lobular hyperplasia and lobular carcinoma in situ: surgical and molecular pathology. Histopathology 35(3):195-200
22. Lynch H T, Lynch J, Conway T, Watson P, Feunteun J, Lenoir G, Narod S, Fitzgibbons R Jr (1994) Hereditary breast cancer and family cancer syndromes. World J Surg 18(1):21-31

1. A method for early detection of a reduced risk of developing cancer, which comprises detecting the absence of a series of genetic polymorphisms associated with a predisposition of developing cancer, including the polymorphisms of the genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267, in a biological sample from the analyzed subject, wherein the absence of the genetic polymorphisms is indicative of significantly decreased risk of developing, at least, breast cancer.

2. The method of claim 1, wherein examined polymorphisms are identified by comparison of the structure of the altered variant with the wild type of the genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267, respectively.

3. (canceled)

4. The method of claim 1, wherein the founder germline variants of genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267 being indicative of a inherited predisposition to cancer are identified from a set or panel of founder mutations of those genes and genetic markers, which are best adjusted for the ethnic population of the investigated human subject.

5. The method of claim 1, wherein the founder germline variants of genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267 being indicative of a inherited predisposition to cancer are identified from a set or panel of founder mutations of those genes and genetic markers, which include all or at least their most frequent variants.

6. The method of claim 1, wherein the germline variant of gene BRCA1 is at least one among C61G, 4153delA and 5382insC, either in homozygous or in heterozygous status, or other BRCA1 variants with analogous functional properties, or sharing a haplotype with the former ones.

7. The method of claim 1, wherein the germline variant of gene BRCA2 is T1915M, either in homozygous or in heterozygous status, or other BRCA2 variants with analogous functional properties, or sharing a haplotype with the former one.

8. The method of claim 1, wherein the germline variant of gene CARD15/NOD2 is 3020insC, either in homozygous or in heterozygous status or other CARD15 variants with analogous functional properties, or sharing a haplotype with the former one.

9. The method of claim 1, wherein the germline variant of gene CDKN2A/P16 is A148T, either in homozygous or in heterozygous status or other CDKN2A variants with analogous functional properties, or sharing a haplotype with the former one.

10. The method of claim 1, wherein the germline variant of gene CHEK2 is at least one among IVS+1G/A, I157T, 1100delC and del5395, either in homozygous or in heterozygous status or other CHEK2 variants with analogous functional properties, or sharing a haplotype with the former ones.

11. The method of claim 1, wherein the germline variant of gene CYP1B1 is a haplotype combination of R48G, A119S and V432L, that has to be present in homozygous status or other CYP1B1 variants with analogous functional properties, or sharing a haplotype with the former ones.

12. The method of claim 1, wherein the germline variant of gene FGFR2/KGFR2 is not homozygous for Adenine at position Rs1219648, or best, is homozygous for Guanine at the same position, or other FGFR2 variants with analogous functional properties, or sharing a haplotype with the former one.

13. The method of claim 1, wherein the germline variant of gene MAP3K1/MEKK1 is any except for the variant homozygous for Adenine at position Rs889312 or other MAP3K1 variants with analogous functional properties, or sharing a haplotype with the former one.

14. The method of claim 1, wherein the germline variant of gene P53/TP53 is R72P either in homozygous or in heterozygous status or other P53 variants with analogous functional properties, or sharing a haplotype with the former one.

15. The method of claim 1, wherein the germline variant of gene TNRC9 is any except for the variant homozygous for Thymine at position Rs3803662 or other TNRC9 variants with analogous functional properties, or sharing a haplotype with the former one.

16. The method of claim 1, wherein the germline variant of gene XPD/ERCC2 is at least one among K751Q and D312N, both in homozygous status, or other XPD variants with analogous functional properties, or sharing a haplotype with the former ones.

17. The method of claim 1, wherein the germline variant of genetic marker Rs6983267 is homozygous for Guanine, or other variants with analogous functional properties, or sharing a haplotype with the former one.

18. The method of claim 1, wherein subjects not carrying none of the mutations among BRCA1 (C61G, 4153delA, 5382insC), BRCA2 (T1915M), CHEK2 (IVS+1G/A, I157T, 1100delC, del5395), CDKN2A (A148T), XPD (D312N, K751Q), P53 (R72P), TNRC9 (Rs3803662 non-TT), FGFR2 (Rs1219648 GG) are statistically significantly at 2-times lower risk for developing breast cancer protected at just 9.4% of the total and healthy controls 16.6%. The odds ratio is 1.9 and is statistically significant.

19. (canceled)

20. The method of claim 1, wherein subjects carrying none of the mutations among BRCA1 (C61G, 4153delA, 5382insC), BRCA2 (T1915M), CHEK2 (IVS+1G/A, I157T, 1100delC, del5395), P53 (R72P), TNRC9 (Rs3803662 non-TT), FGFR2 (Rs1219648 non-AA), CARD15 (3020insC), MAP3K1 (Rs889312 non-AA) are statistically significantly at 19-times lower risk for developing breast cancer of the lobular type.

21. (canceled)

22. The method of claim 1, wherein the mode of detection of germline variants of genes BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267 is based on analysis of DNA, RNA or proteins.

23. The method according to claim 22, wherein DNA or RNA testing is performed using any conventional technique of direct mutation detection, such as sequencing, but more preferably any conventional technique of indirect mutation detection, selected among those such as ASA-, ASO-, RFLP-PCR, Taqman RT-PCR, Maldi-TOF mass-spectrometry or microarray methods.

24-28. (canceled)

29. Composition for prediction of reduced risk of developing cancer, comprising at least 21 different oligonucleotides, one for each of the 12 genetic germline variants analysed, BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267, allowing amplification of those variants 12 regions of the genome of said human subject containing none of said germline variants, preferably comprising all founder mutations, which are characteristic for the ethnic population of the subject, or other variants with analogous properties, as functional markers, or sharing a haplotype with the former ones, as positional markers.

30. (canceled)

31. The method of identification of genetic markers being predictive of significantly reduced predisposition to cancer, characterized by comprising the examination of samples containing genomic DNA from any subject and comparing the frequency of genetic variants within BRCA1, BRCA2, CARD15 (NOD2), CHEK2, CDKN2A (P16), CYP1B1, FGFR2 (KGFR2), MAP3K1 (MEKK1), p53 (TP53), TNRC9, XPD (ERCC2) and the genetic marker Rs6983267, or regions in linkage disequilibrium with them, between examined cancer patients and healthy controls from general population, wherein the absence of a combination of variants significantly overrepresented in patients affected by the specific malignancy is then regarded as genetic marker being predictive of significantly reduced predisposition to cancer.


SEQUENCE LISTING (60 sequences)

SEQ ID NO: 1 - DNA, 22 bp, artificial
  primer B1-5382INSCI1F
cacttccattgaaggaagcttc

SEQ ID NO: 2 - DNA, 20 bp, artificial
  primer B1-5382INSCI1R
tacctttctgtcctggggat

SEQ ID NO: 3 - DNA, 20 bp, artificial
  primer B1-5382INSCI2F
tgacgtgtctgctccacttc

SEQ ID NO: 4 - DNA, 20 bp, artificial
  primer B1-5382INSCI2R
acctttctgtcctggggatt

SEQ ID NO: 5 - DNA, 22 bp, artificial
  primerB1-5382INSCK1F
cacttccattgaaggaagcttc

SEQ ID NO: 6 - DNA, 20 bp, artificial
  primer B1-5382INSCK1R
caaaggggagtggaatacag

SEQ ID NO: 7 - DNA, 20 bp, artificial
  primer B1-5382INSCK2F
atatgacgtgtctgctccac

SEQ ID NO: 8 - DNA, 20 bp, artificial
  primer B1-5382INSCK2R
caaaggggagtggaatacag

SEQ ID NO: 9 - DNA, 20 bp, artificial
  primer B1EX5IK1F
ctcttaagggcagttgtgag

SEQ ID NO: 10 - DNA, 20 bp, artificial
  primer B1EX5IK1R
ttcctactgtggttgcttcc

SEQ ID NO: 11 - DNA, 20 bp, artificial
  primer B1EX5IK2F
atggctcttaagggcagttg

SEQ ID NO: 12 - DNA, 20 bp, artificial
  primer B1EX5IK2R
tgtggttgcttccaacctag

SEQ ID NO: 13 - DNA, 21 bp, artificial
  primer B1_4154DELAI1F
caaaggcatctcaggaacatc

SEQ ID NO: 14 - DNA, 21 bp, artificial
  primer B1_4154DELAI1R
caagcccgttcctctttctca

SEQ ID NO: 15 - DNA, 20 bp, artificial
  primer B1_4154DELAI2F
ttggctcagggttaccgaag

SEQ ID NO: 16 - DNA, 20 bp, artificial
  primer B1_4154DELAI2R
aagcccgttcctctttgtca

SEQ ID NO: 17 - DNA, 20 bp, artificial
  primer B1_4154DELAK1F
ttggctcagggttaccgaag

SEQ ID NO: 18 - DNA, 21 bp, artificial
  primer B1_4154DELAK1R
gtgctccccaaaagcataaac

SEQ ID NO: 19 - DNA, 22 bp, artificial
  primer B1_4154DELAK2F
tcctagccctttcacccataca

SEQ ID NO: 20 - DNA, 21 bp, artificial
  primer B1_4154DELAK2R
gtgctccccaaaagcataaac

SEQ ID NO: 21 - DNA, 26 bp, artificial
  primer b5972F
ctctctagataatgatgaatgatgca

SEQ ID NO: 22 - DNA, 21 bp, artificial
  primer b5972R
ccaaactaacatcacaaggtg

SEQ ID NO: 23 - DNA, 45 bp, artificial
  primer CARD15 F
tccgtcttagctgagtggcgtaggcagaagccctcctgcagggcc

SEQ ID NO: 24 - DNA, 23 bp, artificial
  primer CARD15 R
tcactgaatgtcagaatcagaag

SEQ ID NO: 25 - DNA, 20 bp, artificial
  primer npl6ex2f
aggggtaattagacacctgg

SEQ ID NO: 26 - DNA, 20 bp, artificial
  primer npl6ex2r
tttggaagctctcagggtac

SEQ ID NO: 27 - DNA, 22 bp, artificial
  primer CHEK2ex2/3F
atttatgagcaatttttaaacg

SEQ ID NO: 28 - DNA, 28 bp, artificial
  primer CHEK2ex2/3R
tccagtaaccataagataataatattac

SEQ ID NO: 29 - DNA, 22 bp, artificial
  primer Ch157F
acccatgtatctaggagagctg

SEQ ID NO: 30 - DNA, 24 bp, artificial
  primer Ch157R
ccactgtgatcttctatgtctgca

SEQ ID NO: 31 - DNA, 21 bp, artificial
  primer CHLdel2F
tgtaatgagctgagattgtgc

SEQ ID NO: 32 - DNA, 20 bp, artificial
  primer CHLc2R
cagaaatgagacaggaagtt

SEQ ID NO: 33 - DNA, 18 bp, artificial
  primer CHLdelR
gtctcaaacttggctgcg

SEQ ID NO: 34 - DNA, 20 bp, artificial
  primer CHLcF
ctctgttgtgtacaagtgac

SEQ ID NO: 35 - DNA, 24 bp, artificial
  primer Chk2ex10f
ttaatttaagcaaaattaaatgtc

SEQ ID NO: 36 - DNA, 18 bp, artificial
  primer Chk2ex10r
ggcatggtggtgtgcatc

SEQ ID NO: 37 - DNA, 19 bp, artificial
  primer Chk2delC
tggagtgcccaaaatcata

SEQ ID NO: 38 - DNA, 20 bp, artificial
  primer CYP119F
ctcgttcgctcgcctggcgc

SEQ ID NO: 39 - DNA, 20 bp, artificial
  primer CYP119R
gaagttgcgcatcatgctgt

SEQ ID NO: 40 - DNA, 20 bp, artificial
  primer F1CYP
tccatccagcagaccacgct

SEQ ID NO: 41 - DNA, 19 bp, artificial
  primer R1CYP
gccggacaccacacggaag

SEQ ID NO: 42 - DNA, 20 bp, artificial
  primer CYP1294F
atgcgcttctccagctttgt

SEQ ID NO: 43 - DNA, 20 bp, artificial
  primer CYP1294R
tatggagcacacctcacctg

SEQ ID NO: 44 - DNA, 23 bp, artificial
  primer nfgf F
gcgaatcattgggacaagccatg

SEQ ID NO: 45 - DNA, 20 bp, artificial
  primer nfgf R
tcttccatggtaccggtttc

SEQ ID NO: 46 - DNA, 21 bp, artificial
  probe fgfF1pr
catccttgaagagcgtgtgtc

SEQ ID NO: 47 - DNA, 25 bp, artificial
  primer M3kf F
cccattacttgagatgatctctgag

SEQ ID NO: 48 - DNA, 20 bp, artificial
  primer M3k R
tatgggaaggagtcgttgag

SEQ ID NO: 49 - DNA, 23 bp, artificial
  probe M3kp
ctgctggagaaaggcatgtgcaa

SEQ ID NO: 50 - DNA, 18 bp, artificial
  primer TP53 R72P F
cccggacgatattgaaca

SEQ ID NO: 51 - DNA, 17 bp, artificial
  primer TP53 R72P R
agaagcccagacggaac

SEQ ID NO: 52 - DNA, 23 bp, artificial
  primer Rs6983267 F
ctgaacctgtgggttggctgtca

SEQ ID NO: 53 - DNA, 23 bp, artificial
  primer Rs6983267 R
taataccctcatcgtcctttgag

SEQ ID NO: 54 - DNA, 23 bp, artificial
  primer Ntnrf  F
gcgaatcattgggacaagccatg

SEQ ID NO: 55 - DNA, 25 bp, artificial
  primer Ntnr R
cctaatgattttctctccttaatcc

SEQ ID NO: 56 - DNA, 20 bp, artificial
  probe Ntnr
ctctatagctgtcccttagc

SEQ ID NO: 57 - DNA, 21 bp, artificial
  primer 936gaF
atcaaagagacagacgagcag

SEQ ID NO: 58 - DNA, 21 bp, artificial
  primer 936gaR
gctcaccctgcagcacaacgt

SEQ ID NO: 59 - DNA, 22 bp, artificial
  primer 2253F
ctgttctctccaggaggatcag

SEQ ID NO: 60 - DNA, 23 bp, artificial
  primer 2253R
gacagtgagaaatgtcacctgac